SEQUENCE LISTING (15 sequences)

SEQ ID NO: 1 - DNA, 861 bp, Homo sapiens
  CDS (58)..(717)
  sig_peptide (58)..(174)
  mat_peptide (298)..(717)
aggagggtgggggaacagctcaacaatggctgatgggcgctcctggtgttgatagagatggaacttggacttggaggcctctccacgctgtcccactgcccctggcctaggcggcagcctgccctgtggcccaccctggccgctctggctctgctgagcagcgtcgcagaggcctccctgggctccgcgccccgcagccctgccccccgcgaaggccccccgcctgtcctggcgtcccccgccggccacctgccggggggacgcacggcccgctggtgcagtggaagagcccggcggccgccgccgcagccttctcggcccgcgcccccgccgcctgcacccccatctgctcttccccgcgggggccgcgcggcgcgggctgggggcccgggcagccgcgctcgggcagcgggggcgcggggctgccgcctgcgctcgcagctggtgccggtgcgcgcgctcggcctgggccaccgctccgacgagctggtgcgtttccgcttctgcagcggctcctgccgccgcgcgcgctctccacacgacctcagcctggccagcctactgggcgccggggccctgcgaccgcccccgggctcccggcccgtcagccagccctgctgccgacccacgcgctacgaagcggtctccttcatggacgtcaacagcacctggagaaccgtggaccgcctctccgccaccgcctgcggctgcctgggctgagggctcgctccagggctttgcagactggacccttaccggtggctcttcctgcctgggaccctcccgcagagtcccactagccagcggcctcagccagggacgaaggcctcaaagctgagaggcccctaccggtgggtgatg

SEQ ID NO: 2 - protein, 220 aa, Homo sapiens
MELGLGGLSTLSHCPWPRRQPALWPTLAALALLSSVAEASLGSAPRSPAPREGPPPVLASPAGHLPGGRTARWCSGRARRPPPQPSRPAPPPPAPPSALPRGGRAARAGGPGSRARAAGARGCRLRSQLVPVRALGLGHRSDELVRFRFCSGSCRRARSPHDLSLASLLGAGALRPPPGSRPVSQPCCRPTRYEAVSFMDVNSTWRTVDRLSATACGCLG

SEQ ID NO: 3 - DNA, 2136 bp, Mus musculus
  CDS (975)..(1646)
  sig_peptide (975)..(1091)
  mat_peptide (1215)..(1646)
gcggccgcgaattcggcacgagggcgtctcgctgcagcccgcgatctctactctgcctcctggggtcttctccaaatgtctagcccccacctagagggacctagcctagccagcggggaccggatccggagggtggagcggccaggtgagccctgaaaggtggggcggggcgggggcgctctgggccccaccccgggatctggtgacgccggggctggaatttgacaccggacggcggcgggcaggaggctgctgagggatggagttgggctcggcccccagatgcggcccgcgggctctgccagcaacaagtccctcgggccccagccctcgctgcgactggggcttggagccctgcacccaagggcacagaccggctgccaaggccccacttttaactaaaagaggcgctgccaggtgcacaactctgggcatgatccacttgagcttcgggggaaagcccagcactggtcccaggagaggcgcctagaaggacacggaccaggacccctttggtatggagtgaacgctgagcatggagtggaaggaactcaagttactactttctccaaccaccctggtaccttcagccctgaagtacagagcagaagggtcttagaagacaggaccacagctgtgtgagtctcccccctgaggccttagacgatctctgagctcagctgagctttgtttgcccatctggagaagtgagccattgattgaccttgtggcatcgcgaaggaacaggtcctgccaagcacctaacacagagagcaaggttctccatcgcagctaccgctgctgagttgactctagctactccaacctcctgggtcgcttcgagagactggagtggaaggaggaataccccaaaggataactaactcatctttcagtttgcaagctgccgcaggaagagggtggggaaacgggtccacgaaggcttctgatgggagcttctggagccgaaagctatggaactgggacttgcagagcctactgcattgtcccactgcctccggcctaggtggcagtcagcctggtggccaaccctagctgttctagccctgctgagctgcgtcacagaagcttccctggacccaatgtcccgcagccccgccgctcgcgacggtccctcaccggtcttggcgccccccacggaccacctgcctgggggacacactgcgcatttgtgcagcgaaagaaccctgcgacccccgcctcagtctcctcagcccgcacccccgccgcctggtcccgcgctccagtctcctcccgctgcgctccgcggggcacgcgcggcgcgtgcaggaacccggagcagccgcgcacggaccacagatgcgcgcggctgccgcctgcgctcgcagctggtgccggtgagcgcgctcggcctaggccacagctccgacgagctgatacgtttccgcttctgcagcggctcgtgccgccgagcacgctcccagcacgatctcagtctggccagcctactgggcgctggggccctacggtcgcctcccgggtcccggccgatcagccagccctgctgccggcccactcgctatgaggccgtctccttcatggacgtgaacagcacctggaggaccgtggaccacctctccgccactgcctgcggctgtctgggctgaggatgatctatctccaagcctttgcacactagacccatgtgttgccctacctggaacagctccaccgggcctcactaaccaggagcctcaactcagcaggatatggaggctgcagagctcaggccccaggccggtgagtgacagacgtcgtcggcatgacagacagagtgaaagatgtcggaaccactgaccaacagtcccaagttgttcatggatcccagctctacagacaggagaaacctcagctaaagagaactcctctgggagaatccagaaatggccctctgtcctggggaatgaattttgaagagatatatatacatatatacattgtagtcgcgttgctggaccagcctgtgctgaaaccagtcccgtgttcacttgtggaagccgaagccctatttattatttctaaattatttatttactttgaaaaaaaacggccaagtcggcctccctttagtgagggttaatttgtgatcccggg

SEQ ID NO: 4 - protein, 224 aa, Mus musculus
MELGLAEPTALSHCLRPRWQSAWWPTLAVLALLSCVTEASLDPMSRSPAARDGPSPVLAPPTDHLPGGHTAHLCSERTLRPPPQSPQPAPPPPGPALQSPPAALRGARAARAGTRSSRARTTDARGCRLRSQLVPVSALGLGHSSDELIRFRFCSGSCRRARSQHDLSLASLLGAGALRSPPGSRPISQPCCRPTRYEAVSFMDVNSTWRTVDHLSATACGCLG

SEQ ID NO: 5 - protein, 224 aa, Rattus norvegicus
  SIGNAL (1)..(80)
  mat_peptide (81)..()
MELGLGEPTALSHCLRPRWQPALWPTLAALALLSSVTEASLDPMSRSPASRDVPSPVLAPPTDYLPGGHTAHLCSERALRPPPQSPQPAPPPPGPALQSPPAALRGARAARAGTRSSRARATDARGCRLRSQLVPVSALGLGHSSDELIRFRFCSGSCRRARSPHDLSLASLLDAGALRSPPGSRPISQPCCRPTRYEAVSFMDVNSTWRTVDHLSATACGCLG

SEQ ID NO: 6 - protein, 116 aa, Homo sapiens
AARAGGPGSRARAAGARGCRLRSQLVPVRALGLGHRSDELVRFRFCSGSCRRARSPHDLSLASLLGAGALRPPPGSRPVSQPCCRPTRYEAVSFMDVNSTWRTVDRLSATACGCLG

SEQ ID NO: 7 - protein, 113 aa, Homo sapiens
AGGPGSRARAAGARGCRLRSQLVPVRALGLGHRSDELVRFRFCSGSCRRARSPHDLSLASLLGAGALRPPPGSRPVSQPCCRPTRYEAVSFMDVNSTWRTVDRLSATACGCLG

SEQ ID NO: 8 - protein, 116 aa, Mus musculus
AARAGTRSSRARTTDARGCRLRSQLVPVSALGLGHSSDELIRFRFCSGSCRRARSQHDLSLASLLGAGALRSPPGSRPISQPCCRPTRYEAVSFMDVNSTWRTVDHLSATACGCLG

SEQ ID NO: 9 - protein, 113 aa, Mus musculus
AGTRSSRARTTDARGCRLRSQLVPVSALGLGHSSDELIRFRFCSGSCRRARSQHDLSLASLLGAGALRSPPGSRPISQPCCRPTRYEAVSFMDVNSTWRTVDHLSATACGCLG

SEQ ID NO: 10 - protein, 116 aa, Rattus norvegicus
AARAGTRSSRARATDARGCRLRSQLVPVSALGLGHSSDELIRFRFCSGSCRRARSPHDLSLASLLDAGALRSPPGSRPISQPCCRPTRYEAVSFMDVNSTWRTVDHLSATACGCLG

SEQ ID NO: 11 - protein, 113 aa, Rattus norvegicus
AGTRSSRARATDARGCRLRSQLVPVSALGLGHSSDELIRFRFCSGSCRRARSPHDLSLASLLDAGALRSPPGSRPISQPCCRPTRYEAVSFMDVNSTWRTVDHLSATACGCLG

SEQ ID NO: 12 - protein, 104 aa, Homo sapiens
AAGARGCRLRSQLVPVRALGLGHRSDELVRFRFCSGSCRRARSPHDLSLASLLGAGALRPPPGSRPVSQPCCRPTRYEAVSFMDVNSTWRTVDRLSATACGCLG

SEQ ID NO: 13 - protein, 99 aa, Homo sapiens
GCRLRSQLVPVRALGLGHRSDELVRFRFCSGSCRRARSPHDLSLASLLGAGALRPPPGSRPVSQPCCRPTRYEAVSFMDVNSTWRTVDRLSATACGCLG

SEQ ID NO: 14 - protein, 99 aa, Mus musculus
GCRLRSQLVPVSALGLGHSSDELIRFRFCSGSCRRARSQHDLSLASLLGAGALRSPPGSRPISQPCCRPTRYEAVSFMDVNSTWRTVDHLSATACGCLG

SEQ ID NO: 15 - protein, 99 aa, Rattus norvegicus
GCRLRSQLVPVSALGLGHSSDELIRFRFCSGSCRRARSPHDLSLASLLDAGALRSPPGSRPISQPCCRPTRYEAVSFMDVNSTWRTVDHLSATACGCLG

(19) United States
(12) Patent Application Publication
(10) Pub. No.: US 2008/0260702 A1
(43) Pub. Date: Oct. 23, 2008

(54) Treatment of Retinopathies Using Gfra3 Agonists

(76) Inventor: Jesper Roland Jorgensen, Frederiksberg C (DK)

Correspondence Address: MINTZ, LEVIN, COHN, FERRIS, GLOVSKY AND POPEO, P.C;ATTN: PATENT INTAKE CUSTOMER NO. 30623, ONE FINANCIAL CENTER, BOSTON, MA, 02111, US

(21) Appl. No.: 12/089,867

(22) PCT Filed: Oct. 10, 2006
(86) PCT No.: PCT/DK2006/000572

§ 371 Date: May 13, 2008

(30) Foreign Application Priority Data
Oct. 11, 2005 (DK) PA 2005 01423

(51) Int. Cl. A61K 35/12; A61K 38/02; A61K 38/17; A61P 27/02; C12N 5/06; A61K 31/711

(52) U.S. Cl. 424/93.7; 514/2; 514/44; 514/12; 514/8; 435/375; 435/377

(57) ABSTRACT

The present invention relates to the use of GFRα3 agonists for the treatment of disorders of the retina. A preferred GFRα3 agonist is Neublastin. Neublastin may be administered to the eye using protein formulations, in vivo or ex vivo gene therapy, or implantation of encapsulated cells delivering Neublastin locally to the retina.

The present invention relates to methods and compositions for use in the treatment of disorders of the retina.

### BACKGROUND OF THE INVENTION

Certain neurotrophic factors may promote the survival of photoreceptors. For example, photoreceptors can be rescued to some extent by basic fibroblast growth factor (bFGF) in Royal College of Surgeons (RCS) rats and in albino rats that have been damaged by exposure to constant light (Faktorovich et al., Nature, 347:83-86, 1990). RCS rats have an inherited mutation of a gene expressed in the retinal pigment epithelium (RPE) that results in the failure of the RPE to phagocytize the continuously shed portions of the photoreceptor outer segments and causes photoreceptor degeneration and eventually cell death. A single injection of bFGF into the vitreous body or into the subretinal space, the extracellular space surrounding rods and cones, at the onset of the degeneration transiently rescues photoreceptors (Faktorovich et al., Nature, 347:83-86, 1990). In the tight-damaged model in albino rats, bFGF injected into the subretinal space or the vitreous body two days prior to the onset of constant illumination significantly protects photoreceptors from light injury and prevents cell death (LaVail et al., Proc. Natl. Acad. Sci. USA, 89:11249-11253, 1992). In this model, photoreceptor survival was also seen with acidic FGF (aFGF), brain-derived neurotrophic factor (BDNF), ciliary neurotrophic factor (CNTF), and interleukin-1β. Moderate effects were observed with neurotrophin-3 (NT-3), insulin-like-growth factor II (IGF-II) and tumor necrosis factor-alpha (TNF-alpha). Nerve growth factor (NGF), epidermal growth factor (EGF), platelet-derived growth factor (PDGF) and IGF-I had no effect (LaVail et al., Proc. Natl. Acad. Sci. USA, 89:11249-11253, 1992).

However, many of the factors tested to date induce severe side effects. For example although bFGF is efficacious in the RCS rat and light-induced damage rat models, its therapeutic utility in humans is very limited, due to its hypotensive, mitogenic and potent angiogenic activities. In fact, bFGF injected into the vitreous body causes the invasion of blood-derived macrophages in the inner retina and can produce a massive proliferative vitreoretinopathy (Faktorovich et al., Nature, 347:83-86, 1990). The NGF receptor p75 has also been suggested to mediate cell death under certain conditions (Nykjaer et al, Nature 2004 427:843-48), and mediate pain response following NGF treatment (Mamet J, Lazdunski M, Voilly N, J Biol Chem 2003, 278:48907-13).

Neublastin, also known as Artemin and Enovin, is a 24-kDa homodimeric secreted protein that promotes the survival of neurons of the peripheral and central nervous system (Baudet et al., 2000, Development, 127:4335; Rosenblad et al., 2000, Mol. Cell Neurosci., 15(2):199; GenBank AF120274). The gene encoding neublastin has been cloned and sequenced (Rosenblad et al., 2000, Mol. Cell Neurosci., 15(2):199; Baloh et al., Neuron, 21:1291).

Neublastin is a member of the glial cell line-derived neurotrophic factor (GDNF) ligand family. At the cellular level, GDNF members activate the receptor tyrosine kinase, RET. RET associates with a co-receptor, GDNF family receptor a (GFRα), a glycosylphosphatidyl inositol (GPI) linked membrane protein that provides ligand specificity for RET. Four GFRαs are known (GFRα1-4). Neublastin binds specifically to GFRα3 together with RET forming a ternary signaling complex (Baudet et al. 2000, Development, 127:4335; Baloh et al., 1998, Neuron, 21:1291). Neublastin is not capable of binding to GFRα1 (Carmillo et al., 2005, Biochemistry, 44(7):2545-54) and has never been reported to bind to GFRα2 or 4. GFRα3 is localized predominantly on nociceptive sensory neurons (Orozco et al., 2001, Eur. J. Neurosci., 13(11):2177; Naveilhan et al., 1998, Proc Natl Acad Sci, 95: 11295; Baloh et al., 1998, Proc Natl Acad Sci, 95:5801). These neurons detect pain and injury. Thus, neublastin has clinical application in the general treatment of neuropathy and more specifically in the treatment of neuropathic pain. There are no reports anywhere in the literature of GFRα3 expression in the eye. In a very detailed in situ hybridisation study, Widenfalk et al. (1998, Eur J Neurosci, 10:1508) reported the absence of any GFRα3 expression in developing and developed mouse retina.

Another member of the GDNF family, GDNF, has shown preclinical potential by protecting rat retina from ischemia-reperfusion injury possibly by preventing apoptosis in retinal cells (Wu et al., 2004, Mol Vision, 10:93). Subretinal injections of GDNF protein into P13-P17 rd1 mice resulted in both histological and functional neuroprotection of the rod photoreceptors (Frasson et al., 1999 IOVS 40, 2724-2734). The effect of GDNF is mediated by expression of GFRα1 and 2 in the adult retina (Lindqvist et al., 2004, Exp Neurol, 187(2):487). While GDNF may provide a potential therapeuticum for the treatment of reninopathies, systemic GDNF administration in known to cause side effects in the form of weight toss (Hoane et al, 1999, Cell Tissue Res, 286:179; Keller-Peck et al, 2001, J Neurosci, 21:6136; Lapschak, 1996, Rev Neurosci, 7:165). Local administration of GDNF to the retina using gene therapy vectors may result in transduction of retinal ganglionic cells that project into the brain thereby releasing GDNF into subregions of the brain. As the expression of GFRα1 is abundant in the adult brain GDNF release into the brain may result in undesirable side-effects. Furthermore, the receptor for GDNF, GFRα1, is also found in cornea (You et al., 2001, Invest Opthalmol V is Sci, 42(11):2496). Therefore delivery of GDNF to the eye may result in targeting of both the cornea and the retina.

Accordingly, there is a need for developing therapies for retinal disorders without serious side-effects, to develop more specific therapies for retinal disorders and for providing an alternative to GDNF.

### SUMMARY OF THE INVENTION

In a first aspect the invention relates to the use of a GFRα3 agonist for the preparation of a medicament for the treatment of a disorder of the retina.

In a further aspect the invention relates to a method of treatment of a retinal disorder said method comprising administering to an individual in need thereof a therapeutically effective amount of a GFRα3 agonist.

In a third aspect the invention relates to a method of enhancing the survival of a retinal cell comprising contacting said cell with a GFRα3 agonist.

In a further aspect the invention relates to a method of providing in vitro trophic support to retinal cells, comprising contacting the retinal cells with a GFRα3 agonist in vitro.

The present inventors have determined that GFRα3 is expressed both in the developing mouse and the developed human retina. This is in contrast to reports in literature that do not report GFRα3 in the eye at any developmental stage. The presence of GFRα3 has been verified using both in situ hybridisation and quantitative expression analysis (QRT-PCR). Both experiments have shown that the retina is the only subsection of the eye with significant GFRα3 expression. The QRT-PCR studies have also shown that the amount of GFRα3 expression in retina is comparable to the GFRα3 expression in dorsal root ganglion, a know target for Neublastin therapy.

The realisation that GFRα3 is present in high amounts in the developed human retina mens that administration of a compound capable of forming a complex with GFRα3 and signalling through RET will have an effect on cells of the developed human retina. Therefore GFRα3 agonists can be used for the treatment of disorders resulting in degeneration and/or loss of cells of the developed retina, in particular the human retina.

Compared to GDNF therapy of retinal disorders, the GFRα3 agonist therapies provided herein have the advantage that the GFRα3 co-receptor has a more restricted expression pattern thereby increasing the specificity of the therapy. Numerous papers have reported the absence of GFRα3 in the adult brain. The present inventors have furthermore not been able to detect any GFRα3 expression in other regions of the eye. Therefore a Neublastin therapy comprising local delivery to the eye will result in specific targeting of the retina.

Preferably the GFRα3 agonist is a Neublastin polypeptide, which is known to signal specifically through GFRα3. Other agonists apart from Neublastin may be constructed using domain shuffling between members of the GDNF family. This method has been used to construct GFRα1 agonists (WO 01/47946).

### FIGURES

FIG. 1. In situ hybridisation with two different mGFRα3 probes using transverse sections through the eye of E16 mice as described in Example 1. FIG. 1A shows a section of the eye using the 10× objective. The GFRα3_{—}634-943 probe was used for in situ hybridization. Dark staining in the neural layer of the retina indicates hybridisation of the DIG-labelled probe to GFRα3 mRNA. The stippled box indicates the 40× close-up shown in FIG. 1B. FIG. 1C shows the same section as FIG. 1A, but here the GFRα3_{—}113-377 probe was used for in situ hybridization. The stippled box indicates the 40× close-up shown in FIG. 1D. Note the similar staining patterns for both probes.

FIG. 2. A) GFRα3 temperature gradient RT-PCR on cDNA prepared from Universal Human Reference RNA. Note that one specific 272 bp band was produced at all temperatures without a signal from the negative control, indicating a very efficient and specific primerset. B) GFRα3 standard curve for quantitative real time PCR. Note that crossing-point values (C(T)) of retina and DRG in Table 1 is within the exponential range of the PCR reaction from approximately 10 to 30 cycles.

FIG. 3. Expression of GFRα3 in human tissues analyzed by quantitative RT-PCR as described in Example 2. The result of the analysis using agarose gel electrophoresis is shown. The sizes of the expected products are 272 bp (GFRα3, shown in upper panel) and 240 bp (GAPDH; shown in lower panel). 14 corneal samples were investigated in total, but GFRα3 was not detected in any of them. Only one corneal sample is shown in the figure.

FIG. 4. Immunohistochemistry showing localisation of GFRα3 in E15 rat embryonal eyes. Sections were stained with a GFRα3 antibody (R&D Systems, AF2645). FIG. 4A shows a cross-section of an eye and the surrounding structures. Note the intense staining of trigemina (arrow I). FIG. 4B is an enlarged view of the box in FIG. 4A showing staining of nerve fiber structures in the retinal ganglion cell layer (arrows II) and in the optic nerve (arrow III).

### DETAILED DESCRIPTION

### Definitions

Bioactivity of Neublastin: the ability to bind when dimerised along with GFRα3 to RET and induce RET dimerisation and autophosphorylation. Bioactivity may be measured with Kira Elisa or RET L3 Elisa assays (Carmillo et al, 2005 Biochemistry, 44(7):2545-54). Accordingly a bioactive Neublastin polypeptide activates GFRα3 and substantially does not activate GFRα1, 2, or 4.

“Treatment”, “therapy”, and “medical use” may cover prophylaxis. “Treatment”, “therapy” and “medical use” may also cover inhibition of a disease or disorder, protection against a disease or disorder, and/or prevention (not absolute) of a disease or disorder, and/or prevention of the progression of a disease or disorder. “Treatment”, “therapy” and “medical use” may also include curative, ameliorative, and/or symptomatic treatment, therapy and medical use.

### Retinal Disorders

In mammals, a number of ophthalmic neurodegenerative conditions or diseases involve injury or degeneration of photoreceptors. Trophic factors capable of promoting the survival or regeneration of these neurons would provide useful therapies for the treatment of such diseases.

Photoreceptors are a specialized subset of retinal neurons, that are responsible for vision.

Photoreceptors consist of rods and cones which are the photosensitive cells of the retina. Each rod and cone elaborates a specialized cilium, referred to as an outer segment, that houses the phototransduction machinery. The rods contain a specific light-absorbing visual pigment, rhodopsin. There are three classes of cones in humans, characterized by the expression of distinct visual pigments: the blue cone, green cone and red cone pigments. Each type of visual pigment protein is tuned to absorb light maximally at different wavelengths. The rod rhodopsin mediates scotopic vision (in dim light), whereas the cone pigments are responsible for photopic vision (in bright light). The red, blue and green pigments also form the basis of color vision in humans. The visual pigments in rods and cones respond to light and generate an action potential in the output cells, the rod bipolar neurons, which is then relayed by the retinal ganglion neurons to produce a visual stimulus in the visual cortex.

In humans, a number of diseases of the retina involve the progressive degeneration and eventual death of photoreceptors, leading inexorably to blindness. Degeneration of photoreceptors, such as by inherited retinal dystrophies (e.g., retinitis pigmentosa), age-related macular degeneration and other maculopathies, or retinal detachment, are all characterized by the progressive atrophy and loss of function of photoreceptor outer segments. In addition, death of photoreceptors or loss of photoreceptor function results in partial deafferentation of second order retinal neurons (rod bipolar cells and horizontal cells) in patients with retinal dystrophies, thereby decreasing the overall efficiency of the propagation of the electrical signal generated by photoreceptors. Trophic factors that are capable of rescuing photoreceptors, bipolar cells, horizontal cells, amacrine cells or ganglion cells from cell death and/or restoring the function of dysfunctional (atrophic or dystrophic) photoreceptors, bipolar cells, horizontal cells, amacrine cells or ganglion cells may represent useful therapies for the treatment of such conditions.

In some embodiments the administration of a GFRα3 agonist to the retina results in normalization of pathological changes of a retinal cell, or enhancing retinal cell survival.

By “normalization of pathological changes of a retinal cell” is meant that the GFRα3 agonist induces a change in one or more of the following cellular parameters: expression level of a structural protein, a neurotrophic factor receptor, an ion channel, or a neurotransmitter, or, induces a change in cellular morphology, in each case so as to substantially restore such parameter to the level thereof in a retinal cell of the same or similar phenotype that is unaffected by disease, degeneration, insult, or injury. The normalization of pathological changes of a retinal cell can be monitored immunohistochemically, or by assessing changes in the levels of secreted or shed cellular products, or by assessing in vivo changes in behavior physiologically attributable to function of the affected retinal cell(s).

By “enhancing retinal cell survival” is meant extending the survival of an affected retinal cell beyond the survival period observed in a corresponding retinal cell affected by the same type of disease, disorder, insult, or injury but not treated with an GFRα3 agonist.

### Administration of GFRα3 Agonists to the Retina

In general, devices resulting in tong term release of Neublastin (e.g. encapsulated cells secreting Neublastin or slow release compositions) are implanted into the vitreus humor of the eye to obtain administration of Neublastin to the retina. Devices are preferably inserted into the pars planum of the vitreous humor.

For in vivo gene therapy, gene therapy compositions may be administered to the vitreous humor or to the subretinal space. The tatter wilt result in targeting the retinal epithelial cells, which wilt subsequently secrete Neublastin thereby administering Neublastin to the retina.

Protein compositions other than slow release compositions may also be used, in which case the composition is injected into the vitreous humour. As several injections are most probably required to achieve a clinical result, this delivery method is less preferred compared to the use of in vivo gene therapy, encapsulated cell biodelivery and the use of slow release compositions of Neublastin protein.

Retinopathy, e.g. diabetic retinopathym, is characterized by angiogenesis and retinal degeneration. Retinopathy includes, but is not limited to, diabetic retinopathy, proliferative vitreoretinopathy, and toxic retinopathy. Retinopathies may be treated by delivering Neublastin intraocularly, preferably in the vitreous. We most prefer delivery into the vitreous for this indication. It may also be desirable to co-deliver one or more anti-angiogenic factors intraocularly, preferably intravitreally.

Uveitis involves inflammation and secondary degeneration that may affect retinal cells. This invention contemplates treating retinal degeneration caused by uveitis, preferably by vitreat or anterior chamber administration of Neublastin.

Retinitis pigmentosa, by comparison, is characterized by primary retinal degeneration. This invention contemplates treating retinitis pigmentosa by intraocular, preferably vitreat, administration of Neublastin.

Age-related macutar degeneration involves both angiogenesis and retinal degeneration. Age-related macutar degeneration includes, but is not limited to, dry age-related macutar degeneration, exudative age-related macutar degeneration, and myopic degeneration. This invention contemplates treating this disorder by delivering Neublastin intraocularly, preferably to the vitreous, and/or one or more anti-angiogenic factors intraocularly or periocularly.

Glaucoma is characterized by increased ocular pressure and loss of retinal ganglion cells. Treatments for glaucoma contemplated in this invention include delivery of Neublastin that protect retinal cells from glaucoma associated damage, delivered intraocularly, preferably intravitreally.

Ocular neovascularization is a condition associated with many ocular diseases and disorders and accounting for a majority of severe visual loss. For example, we contemplate treatment of retinal ischemia-associated ocular neovascularization, a major cause of blindness in diabetes and many other diseases; corneal neovascularization, which predisposes patients to corneal graft failure; and neovascularization associated with diabetic retinopathy, central retinal vein occlusion, and possibly age-related macular degeneration.

In one embodiment of the present invention, living cells are encapsulated and surgically inserted (under retrobutbar anesthesia) into the vitreous of the eye. For vitreat placement, the device may be implanted through the sclera, with a portion of the device or tether protruding through the sclera. Most preferably, the entire body of the device is implanted in the vitreous, with no portion of the device protruding into or through the sclera. Preferably the device is tethered to the sclera (or other suitable ocular structure). The tether may comprise a suture eyelet, or any other suitable anchoring means (see e.g. U.S. Pat. No. 6,436,427). The device can remain in the vitreous as long as necessary to achieve the desired prophylaxis or therapy. Such therapies for example include promotion of neuron or photoreceptor survival or repair, or inhibition and/or reversal of retinal neovascutarization, as well as inhibition of uveal, retinal, and optic nerve inflammation. This embodiment is preferable for delivering Neublastin to the retina. Devices for slow release of Neublastin protein may be implanted in substantially the same way.

### Models of Retinopathies

In several in vivo and in vitro models of retinopathies with different underlying causes of retinal neuronal death effects of neurotrophic factors have been described. These in vitro and in vivo models may be used to verify the utility of GFAα3 agonists in the treatment of retinopathies.

In humans, mutations in several genes have been shown to cause photoreceptor degeneration resulting in visual loss and blindness. Examples include genes encoding the rod β-subunit of the cyclic GMP phosphodiesterase, rhodopsin and peripherin. Transgenic mice carrying such mutations have been generated and may be used to test neurotrophic factors in vivo for effect in photoreceptor diseases including retinis pigmentosa and macular degeneration (Portera-Cailliau et al., 1994 PNAS 91, 974-978). Alternatively, testing may be carried out using retinal cell cultures derived from the transgenic mice carrying the appropriate mutation.

The rd1 retinal degeneration mouse, exhibits a rapid retinal degeneration initiated by a recessive mutation of the gene encoding the rod 1-subunit of the cyclic GMP phosphodiesterase. The phenotype and genetic mutation correspond well to the some of forms of Retinitis Pigmentosa (RP) and this mouse model has been widely studied for more than 70 years.

The rd1 mice develop fully differentiated photoreceptors during the second postnatal week, but then suffer nearly complete loss of the rod photoreceptors in the next week, followed by a slower loss of cone photoreceptors (Carter-Dawson et al., 1978 IOVS 17, 489-498).

Protective effects of various neurotrophic factors on the photoreceptors have been described in various experimental set-ups using the rd1 mouse. Subretinal injections of GDNF protein into P13-P17 rd1 mice resulted in both histological and functional neuroprotection of the rod photoreceptors (Frasson et al., 1999 IOVS 40, 2724-2734). In organ cultures using retinal explants from neonatal rd1 retina, addition of tens epithelium-derived growth factor (LEDGF), BDNF and CNTF rescued photoreceptors to different extent (Caffé et al., 2001 IOVS 42, 275-282; Ahuja et al 2001 Neuroreport 12, 2951-2955).

Another widely used transgenic model to study photoreceptor degeneration is the Royal College of Surgeons (RCS) rat which carries a mutation in the MERTK gene that renders RPE cells unable to phagocytose shed photoreceptor outer segments at a normal rate. This ultimately leads to photoreceptor death and visual dysfunction. Using tocal delivery of CNTF to the retina, the degenerating photoreceptors in the RCS rat may be rescued (Huang et al., 2004 J Biomed Sci. 11, 37-48).

Intravitreal injections of AAV expressing GDNF have been shown to protect rat retina from ischemia-reperfusion injury by moderate preservation of the inner retina (Wu et al. 2004 Molecular Vision 10, 93-102). Although poorly understood, glutamate toxicity is thought to play major role in retinal ischemia following acute vascular occlusion. To test for neuroprotective activity against excitotoxic damage, dissociated cultures established from retina treated with glutamate, NMDA or kainic acid may be used (Luo et al., 2004 IOVS 45, 4576-82).

Retinal detachment (RD) is a common cause of visual impairment that results in loss of photoreceptors. In an in vivo model, delivery of GDNF using an AAV vector has been shown to protect against RD-induced photoreceptor damage (Wu et al., 2002 IOVS 43, 3480-3488).

### Neublastin

Neublastin polypeptides are proteins, which promote survival, maintain phenotypic differentiation, prevent degeneration, promote regeneration, and restore the activity of neuronal cells and tissues. Neublastin (initially described, e.g., in WO 00/01815) has alternately been referred to as “artemin” (see, e.g., WO 00/18799) and “enovin” (see, e.g., WO 00/04050).

Neublastin has been classified as a distant member of the TGF-βsuperfamily (Massague, et al., 1994, Trends in Cell Biology, 4: 172-178) and is a member of glial cell line-derived neurotrophic factor ligand family (“GDNF”; WO 93/06116), in the family which includes GDNF, persephin (“PSP”; Milbrandt et al., 1998, Neuron 20: 245253) and neurturin (“NTN”; WO 97/08196). The ligands of the GDNF subfamily have in common their ability to induce signalling through the RET receptor tyrosine kinase. These four ligands of the GDNF subfamily differ in their relative affinities for a family of neurotrophic receptors, the GFRα receptors. Neublastin acts specifically through the GFRα3—RET complex. Baudet et al., Development, 127, pp. 4335-44 (2000); Baloh et al., Neuron, 21, pp. 1291-1302 (1998); Airaksinen et al., Mol. Cell. Neuroscience, 13, pp. 313-325 (1999); Carmillo et al, 2005 Biochemistry, 44(7):2545-54.

An amino acid sequence comparison of neublastin (SEQ ID NO: 2) to the GDNF subfamily members Neurturin, Persephin and GDNF is shown in Table 1. Neublastin polypeptides useful in this invention preferably hold the GDNF subfamily fingerprint, i.e. the amino acid residues underlined in Table 1.

TABLE 1

Amino Acid Sequence Comparison of neublastin to Persephin,
Neurturin, and GDNF

| | |
|---|---|
| Neublastin | MELGLGGLSTLSHCPWPRRQPALWPTLAALALLSSVAEASLGSAPRSPAPREGPPP |
| Neurturin | --------------------MQRWKAAALASVLCSSVLSIWMCREGLLLSHRLGPA |
| Persephin | -------------------------------------------------------- |
| GDNF | -----MKLWDVVAVCLVLLHTASAFPLPAGKRPPEAPAEDRSLGRRRAPFALSSDS |
| Neublastin | VLASPAGHLPGGRTARWCSGRARRPPPQPSRPAPPPPAPPSALPRGGRAARAGGPF |
| Neurturin | LVPLHRLPRTLDARIARLAQYRALLQGAPDAMELRELTPWAGRPPGPRRRAGPRRR |
| Persephin | -MAVGKFLLGSLLLLSLQLGQGWGPDARGVPVADGEFSSEQVAKAGGTWLGTHRPL |
| GDNF | NMPEDYPDQFDDVMDFIQATIKRLKRSPDKQMAVLPRRERNRQAAAANPENSRGKG |
| Neublastin | SRARAAGARGCRLRSQLVPVRALGLGHRSDELVRFRFCSGSCRR-ARSPHDLSLAS |
| Neurturin | RARARLGARPCGLRELEVRVSELGLGYASDETVLFRYCAGACEA-AARVYDLGLRR |
| Persephin | ARLRRALSGPCQLWSLTLSVAELGLGYASEEKVIFRYCAGSCPRGARTQHGLALAR |
| GDNF | RRGQRGKNRGCVLTAIHLNVTDLGLGYETKEELIFRYCSGSCDA-AETTYDKILKN |
| | * * : * ****: :.* : **:*:*:* * :. * |
| Neublastin | LLGAGALRPPPGSRPVSQPCCRPTRYE-AVSFMDVNSTWRTVDRLSATACGCLG |
| Neurturin | LRQRRRLRRE---RVRAQPCCRPTAYEDEVSFLDAHSRYHTVHELSARECACV- |
| Persephin | LQGQGRAHGG--------PCCRPTRYT-DVAFLDDRHRWQRLPQLSAAACGCGG |
| GDNF | LSRNRRLVSD----KVGQACCRPIAFDDDLSFLDDNLVYHILRKHSAKRCGCI- |
| | * .**** : ::*:* . :: : . ** *.* |

*indicates positions which have a single, fully conserved residue.
:indicates that one of the following ‘strong’ groups is fully conserved: -STA, NEQK, NHQK, NDEQ, QHRK, MILV, MILF, HY, FYW.
.indicates that one of the following ‘weaker’ groups is fully conserved: -CSA, ATV, SAG, STNK, STPA, SGND, SNDEQK, NDEQHK, NEQHRK, VLIM, HFY.

From the amino acid sequence alignment shown in Table 1, it can be seen that neublastin has seven cysteine residues at locations that are conserved within the TGF-β superfamily. Based on this sequence alignment, neublastin was shown to be a member of the GDNF subfamily of neurotrophic factors (LGLG-FRFCSGSC-QPCCRP-SATACGC, the GDNF subfamily fingerprint, underlined in Table 1).

The neublastin polypeptides useful herein may be provided in any bioactive form, including the form of pre-proteins, mature proteins, glycosylated proteins, phosphorylated proteins, truncated forms, or any other post-translationally modified protein. It is assumed that a bioactive neublastin is in the dimerized form for each NBN variant, because dimer formation is required for activity. Little to no activity is observed in a monomeric NBN polypeptide. A bioactive neublastin polypeptide includes a dimerized polypeptide that, in the presence of RET, binds to GFRα3 or binds to a complex of GFRα3 and RET, induces dimerization of RET, and autophosphorylation of RET. Accordingly, a “neublastin polypeptide,” as used herein, is a polypeptide which possesses neurotrophic activity (e.g., as described in WO 00/01815).

Neublastin in bioactive form can be detected using the RetL3 ELISA assay and the Kira Elisa assay (Carmillo et al, 2005, Biochemistry, 44(7):2545). Neublastin without biological function will not be detected by the RetL3 ELISA assay.

The following full-length sequences represent the wild type pre-pro neublastin with wild type signal peptide. The native signal peptide of human, mouse and rat neublastin is represented by the first 39 amino acids.
AA-80-AA140 of SEQ ID NO: 2 (“wild type” human prepro),
AA-80-AA144 of SEQ ID No. 4 (mouse prepro),
AA-80-AA144 of SEQ ID NO: 5 (rat prepro),

The following “wild-type” neublastin amino acid (“aa” or “AA”) sequences are exemplary of those that are useful in the methods and compositions of this invention:
AA-AA140 of SEQ ID NO: 2 (mature 140AA; hereafter “140NBN”),
AA25-AA140 of SEQ ID NO: 2 (mature 116AA; hereafter “116NBN”),
AA28-AA140 of SEQ ID NO: 2 (mature 113AA (SEQ ID No. 7); hereafter “113NBN”),
AA1-AA 44 of SEQ ID NO: 4 (mouse mature 144 AA),
AA29-AA144 of SEQ ID NO: 4 (mouse mature 116AA),
AA32-AA144 of SEQ ID NO: 4 (mouse mature 113AA),
AA-AA144 of SEQ ID NO: 5 (rat mature—144 AA),
AA29-AA144 of SEQ ID NO: 5 (rat mature 116AA),
AA32-AA144 of SEQ ID NO: 5 (rat mature 113AA),

In one embodiment, the preferred neublastin polypeptide contains the seven cysteines conserved as in SEQ ID NO. 2 at positions 43, 70, 74, 107, 108, 136, and 138. These seven conserved cysteine residues are known within the TGF-β superfamily to form three intramonomeric disulfide bonds (e.g., in SEQ ID No. 2 between cysteine residues 43-108, 70-136, and 74-138) and one intermonomeric disulfide bond (e.g., in SEQ ID NO. 2 between cysteine residues 107-107), which together with the extended beta strand region constitutes the conserved structural motif for the TGF-βsuperfamily. See, e.g., Daopin et al., Proteins 1993, 17: 176-192.

Preferably the neublastin polypeptide is one of the mature forms or an N-terminally truncated form of the wild type protein.

Neublastin polypeptides useful in the present invention also include truncated forms of the full-length neublastin molecule. In such truncated molecules, one or more amino acids have been deleted from the N-terminus or the C-terminus, preferably the N-terminus. The truncated neublastin polypeptide may be obtained by providing a mature neublastin polypeptide and contacting the mature neublastin polypeptide with at least one protease under conditions sufficient to produce the truncated neublastin polypeptide (WO 02/072826). The truncated Neublastin may also be obtained by directly expressing the truncated form thereby obviating the need for further proteolytical processing (WO 2004/108760).

The truncated neublastin polypeptides described herein preferably include a polypeptide sequence that encompasses the seven cysteine residues conserved in the mature neublastin sequence. In certain preferred embodiments, the truncated neublastin polypeptide includes at least the 85 carboxy terminal amino acids of mature 113NBN neublastin polypeptide. In more preferred embodiments the truncated neublastin polypeptide includes at least the 98 carboxy terminal amino acids of mature human 113 NBN.

One truncated form includes the 97 amino acids from the first to the last of the seven cysteine residues of mature neublastin. This corresponds to amino acids no 2 to 97 of SEQ ID No 13.

Other variants of neublastin include truncated NBN forms. Examples of these include:

(i) the 112AA polypeptide sequence designated herein as NBN 112, which possesses the carboxy terminal 112 amino acids of a mature neublastin polypeptide, e.g., amino acids 29-140 of SEQ ID NO. 2.
(ii) the 111 AA polypeptide sequence designated herein as NBN111, which possesses the carboxy terminal 111 amino acids of a mature neublastin polypeptide, e.g., amino acids 30-140 of SEQ ID NO. 2
(iii) the 110 AA polypeptide sequence designated herein as NBN110, which possesses the carboxy terminal 110 amino acids of a mature neublastin polypeptide, e.g., amino acids 31-140 of SEQ ID NO. 2
(iv) the 109 AA polypeptide sequence designated herein as NBN109, which possesses the carboxy terminal 109 amino acids of a mature neublastin polypeptide, e.g., amino acids 32-140 of SEQ ID NO. 2
(v) the 108AA polypeptide sequence designated herein as NBN108, which possesses the carboxy terminal 108 amino acids of a mature neublastin polypeptide, e.g., amino acids 33-140 of SEQ ID NO. 2
(vi) the 107AA polypeptide sequence designated herein as NBN107, which possesses the carboxy terminal 107 amino acids of a mature neublastin polypeptide, e.g., amino acids 34-140 of SEQ ID NO. 2
(vi) the 106AA polypeptide sequence designated herein as NBN106, which possesses the carboxy terminal 106 amino acids of a mature neublastin polypeptide, e.g., amino acids 35-140 of SEQ ID NO. 2
(viii) the 105AA polypeptide sequence designated herein as NBN105, which possesses the carboxy terminal 105 amino acids of a mature neublastin polypeptide, e.g., amino acids 36-140 of SEQ ID NO. 2
(ix) the 104AA polypeptide sequence designated herein as NBN104, which possesses the carboxy terminal 104 amino acids of a mature neublastin polypeptide, e.g., amino acids 37-140 of SEQ ID NO. 2 (also set forth as SEQ ID No. 12)
(x) the 103AA polypeptide sequence designated herein as NBN103, which possesses the carboxy terminal 103 amino acids of a mature neublastin polypeptide, e.g., amino acids 38-140 of SEQ ID NO. 2
(xi) the 102AA polypeptide sequence designated herein as NBN 102, which possesses the carboxy terminal 102 amino acids of a mature neublastin polypeptide, e.g., amino acids 39-140 of SEQ ID NO. 2
(xii) the 101AA polypeptide sequence designated herein as NBN101, which possesses the carboxy terminal 101 amino acids of a mature neublastin polypeptide, e.g., amino acids 40-140 of SEQ ID NO. 2
(xiii) the 100AA polypeptide sequence designated herein as NBN100, which possesses the carboxy terminal 100 amino acids of a mature neublastin polypeptide, e.g., amino acids 41-140 of SEQ ID NO. 2
(xiv) the 99AA polypeptide sequence designated herein as NBN99, which possesses the carboxy terminal 99 amino acids of a mature neublastin polypeptide, e.g., amino acids 42-140 of SEQ ID NO. 2 (also set forth as SEQ ID No. 13)

In most preferred embodiments, the truncated neublastin polypeptide is the 99 aa, 100 aa, 101 aa, 102 aa, 103 aa, 104 aa, 105 aa, 106 aa, 107 aa, 108 aa, 109 aa, 110 aa, 111 aa or 112 aa carboxy terminal amino acids of mature 113 AA neublastin polypeptide (i.e., NBN99, NBN100, NBN101, NBN102, NBN103, NBN104, NBN105, NBN106, NBN107, NBN108, NBN109, NBN110, NBN111 or NBN112, respectively). These most preferred examples of truncated NBN forms are bioactive (referred to “bioactive truncated neublastin polypeptides”) as they have been demonstrated to have neurotrophic activity. As stated above, NBN dimerization is required for bioactivity, as little to no activity is observed with the NBN monomeric polypeptide.

Truncated forms of the mouse and rat neublastins are also contemplated. These may consist of the C-terminal 99, 100, 101, 102, 103, 104, 105, 106, 107, 108, 109, 110, 111, 112, 113, 114, or 115 amino acids of SEQ ID No 9 (mouse) or they may consist of the C-terminal 99, 100, 101, 102, 103, 104, 105, 106, 107, 108, 109, 110, 111 or 112 amino acids of SEQ ID No 11 (rat).

The NBNs useful in this invention also include those NBN polypeptides that have an amino acid sequence with substantial similarity or identity to the various prepro, pro, mature and truncated “neublastin” polypeptides set forth above. Preferably, the neublastin polypeptide used has at least 70%, more preferably at least 75%, more preferably at least 80%, more preferably 85%, still more preferably 90%, or still further preferably 95% identity or similarity to the mature peptide of the neublastin polypeptides in SEQ ID NO. 2 and 4-11. Most preferably the neublastin polypeptide used has at least 99% similarity or identity to SEQ ID No. 13.

A high level of sequence identity indicates likelihood that the first sequence is derived from the second sequence. Amino acid sequence identity requires identical amino acid sequences between two aligned sequences. Thus, a candidate sequence sharing 70% amino acid identity with a reference sequence, requires that, following alignment, 70% of the amino acids in the candidate sequence are identical to the corresponding amino acids in the reference sequence. Identity is determined by computer analysis, such as, without limitations, the ClustalX computer alignment program (Thompson J D, Gibson T J, Plewniak F, Jeanmougin F, Et Higgins D G: “The ClustalX windows interface: flexible strategies for multiple sequence alignment aided by quality analysis tools”; Nucleic Acids Res. 1997, 25 (24): 4876-82; Higgins D., Thompson J., Gibson T., Thompson J. D., Higgins D. G., Gibson T. J., 1994. CLUSTAL W: improving the sensitivity of progressive multiple sequence alignment through sequence weighting, position-specific gap penalties and weight matrix choice. Nucleic Acids Res. 22:4673-4680)), and the default parameters suggested therein.

The ClustalW software is available from as a ClustalW WWW Service at the European Bioinformatics Institute http://www.ebi.ac.uk/clustalw

Using this program, the mature (bioactive) part of a query and a reference polypeptide are aligned. The number of fully conserved residues are counted and divided by the length of the reference polypeptide. Using this calculation a Neublastin variant exhibits a degree of identity of at least 70%, more preferably 85%, still more preferably 90%, or still further preferably 95%, most preferably at least 99% with the amino acid sequences presented herein as SEQ ID NO: 13 (human 99NBN), SEQ ID NOS: 14 and 15 (rodent 99NBN).

As noted above, the neublastin polypeptides of the invention include variant polypeptides. In the context of this invention, the term “variant polypeptide” includes a polypeptide (or protein) comprising an amino acid sequence that differs from the mature peptides presented as part of SEQ ID NO. 2 (human NBN), or SEQ ID No. 4 and 5 (rodent NBN), at one or more amino acid positions. Such variant polypeptides include the modified polypeptides described above, as well as conservative substitutions, splice variants, isoforms, homologues from other species, and polymorphisms.

As defined herein, the term “conservative substitutions” denotes the replacement of an amino acid residue by another, biologically similar, residue. Typically, biological similarity, as referred to above, reflects substitutions on the wild type sequence with conserved amino acids.

For example, one would expect conservative amino acid substitutions to have little or no effect on the biological activity, particularly if they represent less than 10% of the total number of residues in the polypeptide or protein. Preferably, conservative amino acid substitutions represent changes in less than 5% of the polypeptide or protein, most preferably less than 2% of the polypeptide or protein.

The neublastin polypeptide in one embodiment comprises up to 15 amino acid substitutions, such as up to 12 amino acid substitutions, such as up to 10 amino acid substitutions, such as up to 8 amino acid substitutions, such as up to 5 amino acid substitutions. For example, when calculated in accordance, e.g., with human 113NBN, most preferred conservative substitutions would represent fewer than three amino acid substitutions in the wild type mature amino acid sequence. In a particularly preferred embodiment, there is a single amino acid substitution in the mature sequence, wherein both the substituted and replacement amino acid are non-cyclic. Other examples of particularly conservative substitutions include the substitution of one hydrophobic residue for another, such as isoleucine, valine, leucine or methionine, or the substitution of one polar residue for another, such as the substitution of arginine for lysine, glutamic for aspartic acid, or glutamine for asparagine, and the like.

The term conservative substitution also includes the use of a substituted amino acid residue in place of an un-substituted parent amino acid residue provided that antibodies raised to the substituted polypeptide also immunoreact with the un-substituted polypeptide.

Modifications of this primary amino acid sequence may result in proteins, which have substantially equivalent activity as compared to the unmodified counterpart polypeptide, and thus may be considered functional analogs of the parent proteins. Such modifications may be deliberate, e.g. as by site-directed mutagenesis, or they may occur spontaneously, and include splice variants, isoforms, homologues from other species, and polymorphisms. Such functional analogs are also contemplated according to the invention.

An alignment of 99 C-terminal amino acids from human (SEQ ID NO: 13), mouse (SEQ ID NO: 14) and rat (SEQ ID NO: 15) is shown below:

CLUSTAL W (1.82) multiple sequence alignment

| | |
|---|---|
| | % % |
| Mouse | GCRLRSQLVPVSALGLGHSSDELIRFRFCSGSCRRARSQHDLSLASLLGAGALRSPPGSR |
| Rat | GCRLRSQLVPVSALGLGHSSDELIRFRFCSGSCRRARSPHDLSLASLLDAGALRSPPGSR |
| Human | GCRLRSQLVPVRALGLGHRSDELVRFRFCSGSCRRARSPHDLSLASLLGAGALRPPPGSR |
| | *********** ****** ****:************************.*****.***** |
| | % |
| Mouse | PISQPCCRPTRYEAVSFMDVNSTWRTVDHLSATACGCLG |
| Rat | PISQPCCRPTRYEAVSFMDVNSTWRTVDHLSATACGCLG |
| Human | PVSQPCCRPTRYEAVSFMDVNSTWRTVDRLSATACGCLG |
| | *:**************************:********** |

Substitutions are preferably conducted at positions of non-
conservancy marked with “no star”, “.” or “:”. Other preferred
positions for substitution are denoted with “%”.

Biologically active forms of truncated neublastin are known from WO 02/072826. Truncated and mutated neublastin molecules are also known from WO 02/060929, especially mutated neublastin comprising an amino acid sequence derived from amino acids 8-113 of SEQ ID No. 7, wherein the variant neublastin polypeptide includes one or more of the amino acid substitutions selected from the group consisting of: an amino acid other than arginine at position 14 in the amino acid sequence of said variant polypeptide, an amino acid other than arginine at position 39 in the amino acid sequence of said variant polypeptide, an amino acid other than arginine at position 68 of said variant polypeptide, and an amino acid other than asparagine at position 95 of said variant polypeptide, wherein the positions of said amino acids are numbered in accordance with the polypeptide sequence of SEQ ID NO: 7. The mutated forms may be truncated as described above or include the whole length of the mature protein (amino acids 1-113 of SEQ ID NO 7). Preferably the amino acid at position 14, 39 or 68 is a lysine. These positions may be used for conjugation of a natural or non-natural polymer to increase the serum half-life of the conjugated polypeptide.

In one embodiment the Neublastin variant has substitutions at one or more selected arginine residues of the Neublastin polypeptide. These mutations result in a variant Neublastin polypeptide having reduced or absent heparin binding ability as compared to wild type Neublastin. A variant Neublastin polypeptide contains an amino acid substitution, relative to SEQ ID NO:7, at an arginine residue at one or more of positions 48, 49, and 51.

An arginine residue at position 48, 49, and/or 51 can be substituted with a non-conservative amino acid residue (e.g., glutamic acid) or a conservative or amino acid residue. As detailed in WO 2006/023781, substitution of Arg48, Arg 49, and/or Arg 51 with a non-conservative amino acid can result in a variant Neublastin polypeptide that has reduced heparin binding activity but retained (or even enhanced) Neublastin biological activity.

### Polymer-Conjugated, Glycosylated Neublastin Dimers

In one embodiment a Neublastin polypeptide for retinal therapy is coupled to a naturally or a non-naturally occurring polymer. Such polymer-conjugated Neublastins may have extended serum half-lives compared to non-cunjugated Neublastin. In another embodiment, such polymer-conjugated Neublastin is also glycosylated.

Polymer-Conjugated, Glycosylated Neublastin Dimers display enhanced bioavailability and/or longer serum half-life relative to the corresponding dimer without the combination of polymer conjugation and glycosylation. The polymer-conjugated, glycosylated dimer may also display significantly increased potency in vivo, relative to the potency of the corresponding polypeptide without polymer conjugation and glycosylation.

Neublastin polypeptides in dimers of the invention may be products of a protease cleavage reaction or a chemical cleavage reaction, or may be expressed directly from recombinant DNA construct. Alternatively, they can be chemically synthesized, e.g., using a commercial, solid phase synthesizer.

In preferred embodiments, the polymer comprises a polyalkylene glycol moiety, e.g., polyethylene glycol moiety (PEG).

In preferred embodiments, the polyalkylene glycol moiety is coupled to an amine group of the neublastin polypeptide, or a lysine in a variant neublastin polypeptide. Coupling can occur via an N-hydroxylsuccinimide (NHS) active ester. The active ester can be, e.g., PEG succinimidyl succinate (SS-PEG), succinimidyl butyrate (SPB-PEG), or succinimidyl propionate (SPA-PEG).

The polyalkylene glycol moiety can be, e.g., carboxymethyl-NHS, norleucine-NHS, SC-PEG, tresylate, aldehyde, epoxide, carbonylimidazole, or PNP carbonate.

In some embodiments, the polyalkylene glycol moiety is coupled to a cysteine group of the neublastin polypeptide or variant neublastin polypeptide. For example, coupling can occur via a mateimide group, a vinylsulfone group, a haloacetate group, and a thiol group.

In some embodiments, the neublastin polypeptide or variant neublastin polypeptide is glycosylated. When the neublastin polypeptide or variant polypeptide is glycosylated, the polymer can be coupled to a carbohydrate moiety of the glycosylated neublastin polypeptide or variant neublastin polypeptide. For example, the polymer can be coupled to the glycosylated neublastin polypeptide or variant neublastin polypeptide following oxidation of a hydrazole group or an amino group of the glycosylated neublastin polypeptide or variant neublastin polypeptide, or oxidation of a reactive group of the polymer.

In various embodiments, the neublastin polypeptide or variant neublastin polypeptide comprises one, two, three, or four polymers, such as PEG moieties.

In some embodiments, the polymer is coupled to the polypeptide at a site on the neublastin that is an N terminus. In some embodiments, the polymer is coupled to the polypeptide at a site in a non-terminal amino acid of the neublastin polypeptide or variant neublastin polypeptide.

In preferred embodiments, the polymer is coupled to a solvent exposed amino acid of the neublastin polypeptide or variant neublastin polypeptide.

In some embodiments, the polymer is coupled to the neublastin polypeptide or variant neublastin polypeptide at a residue selected from the group consisting of the amino terminal amino acid of the variant polypeptide, or at an altered residue at position 14, 39, 68, and position 95 in the amino acid sequence of the neublastin polypeptide or variant polypeptide, the numbers referring to positions in SEQ ID NO: 7, and the residue(s) at position 14, 39, 68, or 95 being replaced by a lysine or cystein residue, preferably a Lysine residue. The corresponding positions in a truncated Neublastin or a variant Neublastin can be found using a ClustaolW alignment.

A polymer-conjugated, glycosylated Neublastin dimer comprises a first neublastin polypeptide and a second neublastin polypeptide, wherein: (a) at least one of the polypeptides is glycosylated; (b) at least one of the polypeptides is conjugated at its N-terminus to a water-soluble synthetic polymer; and (c) neither of the polypeptides is conjugated to a water-soluble synthetic polymer at a position other than the N-terminus.

The amino acid sequence of the first neublastin polypeptide and the second neublastin polypeptide may be the same. The first and the second neublastin polypeptide preferably is NBN104 (SEQ ID NO: 12).

Preferably, the water-soluble synthetic polymer is a polyalkylene glycol. The polyalkylene glycol may be linear or branched. Preferably the polyalkylene glycol moiety is a polyethylene glycol (PEG) moiety.

In a preferred embodiment, the N-terminal amino acid of the first neublastin polypeptide and the N-terminal amino acid of the second neublastin polypeptide each is conjugated to a polyalkylene glycol.

The average total molecular weight of the polyalkylene glycol moiety or moieties conjugated to the dimer may be 10-50 kDa, more preferably 15-45 kDa, more preferably 20-40 kDa.

A preferred polymer-conjugated neublastin polypeptide dimer is a homodimer of NBN104 wherein each monomer is conjugated to a PEG moiety at its amino terminus, and glycosylated at position 95 (“2×20 kDa PEG NBN 104”). In some embodiments, the polypeptide in the dimer consists essentially of amino acids 8-113 of SEQ ID NO:7.

One exemplary method for making a polymer conjugated glycosylated neublastin polypeptide dimer includes providing a glycosylated neublastin dimer, e.g., from a eukaryotic cell, and conjugating at least one polypeptide in the dimer to a water-soluble, synthetic polymer, e.g., a polyalkylene glycol moiety. Methods for producing a polymer conjugated glycosylated neublastin polypeptide dimer are disclosed in greater detail in WO 2004/094592. Methods for preparing polymer-conjugated Neublastin polypeptides are disclosed in WO 02/060929.

The glycosylated neublastin may be provided in any bioactive form, including the form of pre-pro-proteins, pro-proteins, mature proteins, phosphorylated proteins, non-phosphorylated proteins, truncated forms, or any other posttranstational modified protein. In some embodiments, a polypeptide of the invention has the amino acid sequence presented as SEQ ID NO: 2, holding a glycosylated asparagine residue at position 122; or the amino acid sequence presented as SEQ ID NO: 7, holding a glycosylated asparagine residue at position 95, or the analogous position in any neublastin polypeptide when aligned by, e.g., ClustalW computer software.

In general, a dimer isolated from a mammalian cell, or other such cell able to glycosylate proteins, will be glycosylated at amino acid position 95 (SEQ ID NO 7). Methods of glycosylating proteins in vitro are known in the art and may be employed to glycosylate neublastin polypeptides or polypeptide dimers if so desired.

The polymer conjugated to a neublastin polypeptide is water-soluble. Preferably, the polymer is suitable for use in a pharmaceutical composition. Examples of suitable water-soluble polymers include PEG, copolymers of ethylene glycol/propylene glycol, carboxymethylcellulose, dextran, polyvinly alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinly pyrrolidone)-PEG, propropylene glycol hornopolymers, polypropylene oxide/ethylene oxide co-polymers, polyoxyethylated polyols (e.g., glycerol), polyvinylalcohol, and mixtures thereof.

Avarage molecular weight per polymer chain is chosen in accordance with the desired average total molecular weight of the polymer(s) conjugated per dimer, e.g., 10-50 kDa, 15-45 kDa, or 20-40 kDa per dimer. In PEG preparations, some molecules weigh more, some less, than the stated molecular weight. Thus, molecular weight is typically specified as “average molecular weight”. Various conjugation methods are known in the art. See, e.g., EP 0 401 384 (coupling PEG to G-CSF); Malik et. al., Exp. Hematol. 20: 1028-1035, 1992 (PEGylation of GM-CSF using tresyl chloride).

### Neublastin Polynucleotides

Neublastin polynucleotides for use in treating retinal disorders are polynucleotides encoding a bioactive Neublastin polypeptide as described above. Exemplary Neublastin polynucleotides include the coding sequence of SEQ ID No 1 encoding human pre-pro-Neublastin and SEQ ID No 3 encoding mouse pre-pro-Neublatin.

As Neublastin is bioactive in N-terminally truncated form, the Neublastin polynucleotides of the present invention need not include the whole coding sequence of SEQ ID No 1 or 3. Preferably the Neublastin polynucleotide comprises a sequence encoding the N-terminal 99 amino acids of mature Neublastin, corresponding to bases 421-717 of SEQ ID No 1. Included within the scope of the present invention are also variants of this sequence encoding a bioactive Neublastin polypeptide as described above.

Such variants may comprise sequence variants. For example sequence variants may include polynucleotides encoding a bioactive Neublastin, wherein the polynucleotide has at least 70% sequence identity to bases 421-717 of SEQ ID No. 1, more preferably at least 75%, more preferably at least 80%, more preferably at least 85%, more preferably at Least 90%, more preferably at least 95%, more preferably at Least 98% sequence identity to bases 421-717 of SEQ ID NO. 1.

Variants may also be defined with reference to the encoded bioactive Neublastin polypeptide. Preferably, the encoded bioactive Neublastin polypeptide comprises a polypeptide having at least 60% sequence identity to SEQ ID No 13, more preferably at least 65%, more preferably at least 70%, more preferably at least 75%, more preferably at least 80%, more preferably at least 85%, more preferably at least 90%, more preferably at least 95%, more preferably at least 98% sequence identity to SEQ ID NO. 13.

For enhanced expression, the Neublastin coding sequence does not encode functional Neublastin pro-domain. Even more preferably, the Neublastin coding sequence comprises a heterologous signal peptide linked to the sequence coding for mature or truncated Neublastin as described in WO 2004/108760. Pro-less expression constructs may be used for in vivo gene therapy, for ex vivo gene therapy, and for generation of Neublastin secreting therapeutic cells (for use e.g. in encapsulated cell biodelivery as herein described).

The most preferred, pro-less expression constructs encode a chimeric protein consisting of a signal peptide selected from the group consisting of murine immunogobulin signal peptide (MKCSWVIFFLMAVVTGVNS), human growth hormone signal peptide (MATGSRTSLLLAFGLLCLSWLQEGSA), rat albumin signal peptide (MKWVTFLLLLFISGSAFS), and modified rat albumin signal peptide (MKWVTFLLFLLFISGDAFA), and a Neublastin polypeptide selected from the group consisting of mature human Neublastin or an N-terminally truncated human Neublastin, i.e. wherein one or more of amino acids 1 to 14 of SEQ ID NO 7 are missing. The signal peptides are as defined in WO 2004/108760.

### Pharmaceutical Compositions

A Neublastin polypeptide can be incorporated into a pharmaceutical composition containing a therapeutically effective amount of the polypeptide and one or more adjuvants, excipients, carriers, and/or diluents. Acceptable diluents, carriers and excipients typically do not adversely affect a recipient's homeostasis (e.g., electrolyte balance). Acceptable carriers include biocompatible, inert or bioabsorbable salts, buffering agents, oligo- or polysaccharides, polymers, viscosity-improving agents, preservatives and the like. One exemplary carrier is physiologic saline (0.15 M NaCl, pH 7.0 to 7.4). Another exemplary carrier is 50 mM sodium phosphate, 100 mM sodium chloride. Further details on techniques for formulation and administration of pharmaceutical compositions can be found in, e.g., REMINGTON'S PHARMACEUTICAL SCIENCES (Maack Publishing Co., Easton, Pa.).

Administration of a pharmaceutical composition containing a Neublastin polypeptide can be systemic or local. Pharmaceutical compositions can be formulated such that they are suitable for parenteral and/or non-parenteral administration. Specific administration modalities include subcutaneous, intravenous, intramuscular or other systemic administrations. Because the eye is protected by the blood-retina barrier, local administration to the vitreous humor or to the subretinal space is preferred. Local topical administration to the cornea may also result in treatment of retinal cells.

Formulations suitable for parenteral administration conveniently contain a sterile aqueous preparation of the Neublastin polypeptide, which preferably is isotonic with the blood of the recipient (e.g., physiological saline solution). Formulations may be presented in unit dose or multi dose form.

Therapeutically effective amounts of a pharmaceutical composition may be administered to a subject in need thereof in a dosage regimen ascertainable by one of skill in the art. For example, a composition can be administered to the subject, e.g., systemically at a dosage from 0.01 μg/kg to 1000 μg/kg body weight of the subject, per dose. In another example, the dosage is from 1 μg/kg to 100 μg/kg body weight of the subject, per dose. In another example, the dosage is from 1 μg/kg to 30 μg/kg body weight of the subject, per dose, e.g., from 3 μg/kg to 10 μg/kg body weight of the subject, per dose.

Intraocularly, preferably in the vitreous, we contemplate delivery of protein formulations of Neublastin in a dosage range of 5 ng to 50 μg, preferably 10 ng to 10 μg, and most preferably 100 ng to 5 μg per eye per patient per day. These dosages may be achieved by injections, or infusions of protein compositions. For delivery via gene therapy or through the implantation of encapsulated cells delivering Neublastin, the dosages preferably are approximately 100 fold lower.

In order to optimize therapeutic efficacy, a Neublastin polypeptide is first administered at different dosing regimens. The unit dose and regimen depend on factors that include, e.g., the species of mammal, its immune status, the body weight of the mammal. Typically, protein levels in tissue are monitored using appropriate screening assays as part of a clinical testing procedure, e.g., to determine the efficacy of a given treatment regimen.

The frequency of dosing for a Neublastin polypeptide is within the skills and clinical judgement of physicians. Typically, the administration regime is established by clinical trials which may establish optimal administration parameters. However, the practitioner may vary such administration regimes according to the subject's age, health, weight, sex and medical status. The frequency of dosing may also vary between acute and chronic treatments for neuropathy. In addition, the frequency of dosing may be varied depending on whether the treatment is prophylactic or therapeutic.

### Gene Therapy Vectors

Broadly, gene therapy seeks to transfer new genetic material to the cells of a patient with resulting therapeutic benefit to the patient. Such benefits include treatment or prophylaxis of diseases, disorders and other conditions of the retina. The gene therapy vectors of the present invention comprises a Neublastin polynucleotide as described herein and encodes a Neublastin polypeptide as described herein.

Ex vivo gene therapy approaches involve modification of isolated cells, which are then infused, grafted or otherwise transplanted into the patient. In vivo gene therapy seeks to directly target host patient tissue in vivo.

Viruses useful as gene transfer vectors include papovavirus, adenovirus, vaccinia virus, adeno-associated virus, herpesvirus, and retrovirus. Suitable retroviruses include the group consisting of HIV, SIV, FIV, EIAV, MoMLV.

Preferred viruses for treatment of disorders of the retina are lentiviruses and adeno-associated viruses. Both types of viruses can integrate into the genome without cell divisions, and both types have been tested in pre-clinical animal studies for indications of the eye (Wu et al., 2004, Mol Vision, 10:93).

Methods for preparation of AAV are described in the art, e.g. U.S. Pat. No. 5,677,158. Both adenovirus and adeno-associated virus have been approved for clinical phase I trials.

Special and preferred types of retroviruses include the lentiviruses which can transduce a cell and integrate into its genome without cell division. Thus preferably the vector is a replication-defective lentivirus particle. Such a lentivivus particle can be produced from a lentiviral vector comprising a 5′ lentiviral LTR, a tRNA binding site, a packaging signal, a promoter operably linked to a polynucleotide signal encoding said fusion protein, an origin of second strand DNA synthesis and a 3′ lentiviral LTR. Methods for preparation and in vivo administration of lentivirus to neural cells are described in US 20020037281 (Methods for transducing neural cells using lentiviral vectors).

Retroviral vectors are the vectors most commonly used in human clinical trials, since they carry a 7-8 kb which is more than many other viral vectors and since they have the ability to infect cells and have their genetic material stably integrated into the host cell with high efficiency. See, e.g., WO 95/30761; WO 95/24929. Oncovirinae require at least one round of target cell proliferation for transfer and integration of exogenous nucleic acid sequences into the patient. Retroviral vectors integrate randomly into the patient's genome.

Two classes of retroviral particles have been described; ecotropic, which can infect mouse cells efficiently, and amphotropic, which can infect cells of many species. A third class includes xenotrophic retrovirus which can infect cells of another species than the species which produced the virus. Their ability to integrate only into the genome of dividing cells has made retroviruses attractive for marking cell lineages in developmental studies and for delivering therapeutic or suicide genes to cancers or tumors. These vectors may be particularly useful in the central nervous system including the eye, where there is a relative lack of cell division in adult patients.

For use in human patients, the retroviral vectors must be replication defective. This prevents further generation of infectious retroviral particles in the target tissue—instead the replication defective vector becomes a “captive” transgene stable incorporated into the target cell genome. Typically in replication defective vectors, the gag, env, and pol genes have been deleted (along with most of the rest of the viral genome). Heterologous DNA is inserted in place of the deleted viral genes. The heterologous genes may be under the control of the endogenous heterologous promoter, another heterologous promoter active in the target cell, or the retroviral 5′ LTR (the viral LTR is active in diverse tissues). Typically, retroviral vectors have a transgene capacity of about 7-8 kb.

Replication defective retroviral vectors require provision of the viral proteins necessary for replication and assembly in trans, from, e.g., engineered packaging cell lines. It is important that the packaging cells do not release replication competent virus and/or helper virus. This has been achieved by expressing viral proteins from RNAs lacking the φ signal, and expressing the gag/pol genes and the env gene from separate transcriptional units. In addition, in some packaging cell lines, the 5‘LTR’s have been replaced with non-viral promoters controlling expression of these genes and polyadenylation signals have been added. These designs minimize the possibility of recombination leading to production of replication competent vectors, or helper viruses. See, e.g., U.S. Pat. No. 4,861,719, herein incorporated by reference.

### Expression Vectors

Construction of vectors for generation of Neublastin gene therapy vectors or Neublastin secreting cells for use in the invention may be accomplished using conventional techniques which do not require detailed explanation to one of ordinary skill in the art. For review, however, those of ordinary skill may wish to consult Maniatis et al., in Molecular Cloning: A Laboratory Manual, Cold Spring Harbor Laboratory, (NY 1982).

Briefly, construction of recombinant expression vectors employs standard ligation techniques. For analysis to confirm correct sequences in constructed vectors, the ligation mixtures may be used to transfect/transduce a host cell and successful genetically altered cells may be selected by antibiotic resistance where appropriate.

Vectors from the transfected/transduced cells are prepared, analysed by restriction and/or sequenced by, for example, the method of Messing, et al., (Nucleic Acids Res., 9: 309-, 1981), the method of Maxam, et al., (Methods in Enzymology, 65: 499, 1980), the Sanger dideoxy-method or other suitable methods which will be known to those skilled in the art.

Size separation of cleaved fragments is performed using conventional get electrophoresis as described, for example, by Maniatis, et al., (Molecular Cloning, pp. 133-134, 1982).

Expression of a gene is controlled at the transcription, translation or post-translation levels. Transcription initiation is an early and critical event in gene expression. This depends on the promoter and enhancer sequences and is influenced by specific cellular factors that interact with these sequences. The transcriptional unit of many prokaryotic genes consists of the promoter and in some cases enhancer or regulator elements (Banerji et al., Cell 27: 299 (1981); Corden et al., Science 209: 1406 (1980); and Breathnach and Chambon, Ann. Rev. Biochem. 50: 349 (1981)). For retroviruses, control elements involved in the replication of the retroviral genome reside in the long terminal repeat (LTR) (Weiss et al., eds., The molecular biology of tumor viruses: RNA tumor viruses, Cold Spring Harbor Laboratory, (NY 1982)). Moloney mouse leukemia virus (MLV) and Rous sarcoma virus (RSV) LTRs contain promoter and enhancer sequences (Jolly et al., Nucleic Acids Res. 11: 1855 (1983); Capecchi et al., In: Enhancer and eukaryotic gene expression, Gulzman and Shenk, eds., pp. 101-102, Cold Spring Harbor Laboratories (NY 1991). Other potent promoters include those derived from cytomegalovirus (CMV) and other wild-type viral promoters.

Promoter and enhancer regions of a number of non-viral promoters have also been described (Schmidt et al., Nature 314: 285 (1985); Rossi and decrombrugghe, Proc. Natl. Acad. Sci. USA 84: 5590-5594 (1987)). Methods for maintaining and increasing expression of transgenes in quiescent cells include the use of promoters including collagen type I (1 and 2) (Prockop and Kivirikko, N. Eng. J. Med. 311: 376 (1984); Smith and Niles, Biochem. 19: 1820 (1980); de Wet et al., J. Biol. Chem., 258: 14385 (1983)), SV40 and LTR promoters.

According to one embodiment of the invention, the promoter is a constitutive promoter selected from the group consisting of: ubiquitin promoter, CMV promoter, JeT promoter, SV40 promoter, Elongation Factor 1 alpha promoter (EF1-alpha), chick beta-actin, PGK, MT-1 (Metallothionin).

Examples of inducible/repressible promoters include: Tet-On, Tet-Off, Rapamycin-inducible promoter, Mx1, and RU486.

In addition to using viral and non-viral promoters to drive transgene expression, an enhancer sequence may be used to increase the level of transgene expression. Enhancers can increase the transcriptional activity not only of their native gene but also of some foreign genes (Armelor, Proc. Natl. Acad. Sci. USA 70: 2702 (1973)). For example, in the present invention collagen enhancer sequences are used with the collagen promoter 2 (I) to increase transgene expression. In addition, the enhancer element found in SV40 viruses may be used to increase transgene expression. This enhancer sequence consists of a 72 base pair repeat as described by Gruss et al., Proc. Natl. Acad. Sci. USA 78: 943 (1981); Benoist and Chambon, Nature 290: 304 (1981), and Fromm and Berg, J. Mol. Appl. Genetics, 1: 457 (1982), all of which are incorporated by reference herein. This repeat sequence can increase the transcription of many different viral and cellular genes when it is present in series with various promoters (Moreau et al., Nucleic Acids Res. 9: 6047 (1981).

### Pharmaceutical Preparations for Gene Therapy

To form a neublastin gene therapy composition for use in the invention, neublastin encoding expression vectors may be placed into a pharmaceutically acceptable suspension, solution or emulsion. Suitable mediums include saline and liposomal preparations.

More specifically, pharmaceutically acceptable carriers may include sterile aqueous of non-aqueous solutions, suspensions, and emulsions. Examples of nonaqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils.

Intravenous vehicles include fluid and nutrient replenishers, electrolyte replenishers (such as those based on Ringer's dextrose), and the like.

Preservatives and other additives may also be present such as, for example, antimicrobials, antioxidants, chelating agents, and inert gases and the like. Further, a composition of neublastin transgenes may be lyophilized using means well known in the art, for subsequent reconstitution and use according to the invention.

A colloidal dispersion system may also be used for targeted gene delivery. Colloidal dispersion systems include macromolecule complexes, nanocapsules, microspheres, beads, and lipid-based systems including oil-in-water emulsions, micelles, mixed micelles, and liposoms. Liposomes are artificial membrane vesicles which are useful as delivery vehicles in vitro and in vivo. It has been shown that large unilamellar vesicles (LUV), which range in size from 0.2-4.0 μm can encapsulate a substantial percentage of an aqueous buffer containing large macro molecules. RNA, DNA and intact virions can be encapsulated within the aqueous interior and be delivered to cells in a biologically active form (Fraley, et al., Trends Biochem. Sci., 6: 77, 1981). In addition to mammalian cells, liposomes have been used for delivery of operatively encoding transgenes in plant, yeast and bacterial cells. In order for a liposome to be an efficient gene transfer vehicle, the following characteristics should be present: (1) encapsulation of the genes encoding the neublastin at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino, et al., Biotechniques, 6: 682, 1988).

The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs which contain sinusoidal capillaries.

Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted gene delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand.

A further example of a delivery system includes transplantation into the therapeutic area of a composition of packaging cells capable of producing vector particles as described in the present invention. Methods for encapsulation and transplantation of such cells are known in the art, in particular from WO 97/44065. By selecting a packaging cell line capable of producing lentiviral particles, transduction of non-dividing cells in the therapeutic area is obtained. By using retroviral particles capable of transducing only dividing cells, transduction is restricted to de-novo differentiated cells in the therapeutic area.

### Methods for Delivery of Gene Therapy Vector Composition

Following the protocol defined by the invention, direct delivery of a neublastin composition may be achieved by means familiar to those of skill in the art, including microinjection through a surgical incision (see, e.g., Capecchi, Cell, 22: 479-488 (1980)); electropotation (see, e.g., Andreason and Evans, Biotechniques, 6: 650-660 (1988)); infusion, chemical complexation with a targeting molecule or co-precipitant (e.g., liposome, calcium), and microparticle bombardment of the target tissue (Tang, et al., Nature, 356: 152-154 (1992)).

### Encapsulation of Cells

Encapsulated cell biodelivery is based on the concept of isolating cells from the recipient host's immune system by surrounding the cells with a semipermeable biocompatible material before implantation within the host. The invention includes a device in which cells are encapsulated in an immunoisolatory capsule. An “immunoisolatory capsule” means that the capsule, upon implantation into a recipient host, minimizes the deleterious effects of the host's immune system on the cells in the core of the device. Cells are immunoisolated from the host by enclosing them within implantable polymeric capsules formed by a microporous membrane. This approach prevents the cell-to cell contact between host and implanted tissues, eliminating antigen recognition through direct presentation. The membranes used can also be tailored to control the diffusion of molecules, such as Neublastin based on their molecular weight. Using encapsulation techniques, Cells can be transplanted into a host without immune rejection, either with or without use of immunosuppressive drugs. Useful biocompatible polymer capsules usually contain a core that contains cells, either suspended in a liquid medium or immobilized within an immobilizing matrix, and a surrounding or peripheral region of permselective matrix or membrane (“jacket”) that does not contain isolated cells, that is biocompatible, and that is sufficient to protect cells in the core from detrimental immunological attack. Encapsulation reduces entry of elements of the immune system into the capsule, thereby protecting the encapsulated cells from immune destruction. The semipermeable nature of the capsule membrane also permits the biologically active molecule of interest to easily diffuse from the capsule into the surrounding host tissue.

The capsule can be made from a biocompatible material. A “biocompatible material” is a material that, after implantation in a host, does not elicit a detrimental host response sufficient to result in the rejection of the capsule or to render it inoperable, for example through degradation. The biocompatible material is relatively impermeable to large molecules, such as components of the host's immune system, but is permeable to small molecules, such as insulin, growth factors, and nutrients, while allowing metabolic waste to be removed. A variety of biocompatible materials are suitable for delivery of growth factors by the composition of the invention. Numerous biocompatible materials are known, having various outer surface morphologies and other mechanical and structural characteristics.

Preferably the capsule of this invention will be similar to those described by WO 92/19195 or WO 95/05452, incorporated by reference; or U.S. Pat. Nos. 5,639,275; 5,653,975; 4,892,538; 5,156,844; 5,283,187; or U.S. Pat. No. 5,550,050, incorporated by reference. Such capsules allow for the passage of metabolites, nutrients and therapeutic substances while minimizing the detrimental effects of the host immune system. Components of the biocompatible material may include a surrounding semipermeable membrane and the internal cell-supporting scaffolding. Preferably, the genetically altered cells are seeded onto the scaffolding, which is encapsulated by the permselective membrane. The cell-supporting scaffold may be made from any biocompatible material selected from the group consisting of acrylic, polyester, polyethylene, polypropylene polyacetonitrite, polyethylene teraphthalate, nylon, polyamides, polyurethanes, polybutester, silk, cotton, chitin, carbon, or biocompatible metals. Also, bonded fiber structures can be used for cell implantation (U.S. Pat. No. 5,512,600, incorporated by reference). Biodegradable polymers include those comprised of poly(lactic acid) PLA, poly(lactic-coglycolic acid) PLGA, and poly(glycotic acid) PGA and their equivalents. Foam scaffolds have been used to provide surfaces onto which transplanted cells may adhere (WO 98/05304, incorporated by reference). Woven mesh tubes have been used as vascular grafts (WO 99/52573, incorporated by reference). Additionally, the core can be composed of an immobilizing matrix formed from a hydrogel, which stabilizes the position of the cells. A hydrogel is a 3-dimensional network of cross-linked hydrophilic polymers in the form of a get, substantially composed of water.

Various polymers and polymer blends can be used to manufacture the surrounding semipermeable membrane, including polyacrylates (including acrylic copolymers), polyvinytidenes, polyvinyl chloride copolymers, polyurethanes, polystyrenes, polyamides, cellulose acetates, cellulose nitrates, polysutfones (including polyether sulfones), polyphosphazenes, polyacrytonitrites, poly(acrytonitrite/covinyl chloride), as well as derivatives, copolymers and mixtures thereof. Preferably, the surrounding semipermeable membrane is a biocompatible semipermeable hollow fiber membrane. Such membranes and methods of making them are disclosed by U.S. Pat. Nos. 5,284,761 and 5,158,881, incorporated by reference. The surrounding semipermeable membrane is formed from a polyether sulfone hollow fiber, such as those described by U.S. Pat. No. 4,976,859 or U.S. Pat. No. 4,968,733, incorporated by reference. An alternate surrounding semipermeable membrane material is poly(acrylonitrile/covinyl chloride).

The capsule can be any configuration appropriate for maintaining biological activity and providing access for delivery of the product or function, including for example, cylindrical, rectangular, disk-shaped, patch-shaped, ovoid, stellate, or spherical. Moreover, the capsule can be coiled or wrapped into a mesh-like or nested structure. If the capsule is to be retrieved after it is implanted, configurations which tend to lead to migration of the capsules from the site of implantation, such as spherical capsules small enough to travel in the recipient host's blood vessels, are not preferred. Certain shapes, such as rectangles, patches, disks, cylinders, and flat sheets offer greater structural integrity and are preferable where retrieval is desired.

When macrocapsules are used, preferably between 10³ and 10⁸ cells are encapsulated, most preferably 10⁵ to 10⁷ cells are encapsulated in each device. Dosage may be controlled by implanting a fewer or greater number of capsules, preferably between 1 and 10 capsules per patient.

The scaffolding may be coated with extracellular matrix (ECM) molecules. Suitable examples of extracellular matrix molecules include, for example, collagen, laminin, and fibronectin. The surface of the scaffolding may also be modified by treating with plasma irradiation to impart charge to enhance adhesion of cells.

Any suitable method of sealing the capsules may be used, including the use of polymer adhesives or crimping, knotting and heat sealing. In addition, any suitable “dry” sealing method can also be used, as described, e.g., in U.S. Pat. No. 5,653,687, incorporated by reference.

The ARPE-19 cell line is a superior platform cell line for encapsulated cell based delivery technology and is also useful for unencapsulated cell based delivery technology. The ARPE-19 cell line is hardy (i.e., the cell line is viable under stringent conditions, such as implantation in the central nervous system or the intra-ocular environment). ARPE-19 cells can be genetically modified to secrete a substance of therapeutic interest. ARPE-19 cells have a relatively long life span. ARPE-19 cells are of human origin. Furthermore, encapsulated ARPE-19 cells have good in vivo device viability. ARPE-19 cells can deliver an efficacious quantity of growth factor. ARPE-19 cells elicit a negligible host immune reaction. Moreover, ARPE-19 cells are non-tumorigenic.

Methods and apparatus for implantation of capsules into the eye are described in U.S. Pat. No. 6,299,895 (Hammang et al).

### Encapsulated Gene Therapy

In one aspect the invention relates to a biocompatible capsule comprising: a core comprising living packaging cells that secrete a viral vector for infection of a target cell, wherein the viral vector is a vector encoding a Neublastin polypeptide; and an external jacket surrounding said core, said jacket comprising a permeable biocompatible material, said material having a porosity selected to permit passage of retroviral vectors of 100 nm diameter thereacross, permitting release of said viral vector from said capsule.

Preferably, the core additionally comprises a matrix, the packaging cells being immobilized by the matrix. According to one embodiment, the jacket comprises a hydrogel or thermoplastic material.

Examples of suitable cells for packaging cell lines include HEK293, NIH3T3, PG13, and ARPE-19 cells. Preferred cells include PG13 and 3T3 cells.

Methods and devices for encapsulation of packaging cells are disclosed in U.S. Pat. No. 6,027,721 hereby incorporated by reference in its entirety.

### EXAMPLES

### Example 1

### In Situ Hybridisation

A partial mouse GFRα3 sequence was PCR cloned corresponding to the 113-1285 bp region in the sequence deposited in GenBank with accession number NM_{—}010280. As source for the cloning a cDNA library from a tissue known to express GFRα3 may be used. Alternatively a GFRα3 clone can be obtained from the IMAGE consortium ((The I.M.A.G.E. Consortium: “An integrated molecular analysis of genomes and their expression”, Lennon, Auffray, Polymeropoulos, and Soares, [1996], Genomics 33: 151-152) clones obtainable from RZPD, Berlin, Germany (for example IMAGE:475-497)). Correct sequence was verified by DNA sequencing. From this sequence, two different GFRα3 probes were generated for in situ hybridization. The first probe, previously described by Naveilhan et al., 1998, Proc Natl Acad. Sci., is complementary to mGFRα3 bases 634-943 of NM_{—}010280 whereas the second probe is complementary to bases 113-377 of the same sequence. The individual probe sequences were amplified by RT-PCR using the cloned mGFRα3 sequence as template and a reverse primer containing a T7 site for subsequent in vitro transcription. Labelling of the probes with digoxygenin (DIG) was done with the DIG RNA Labelling Kit (SP6/T7) from Roche according to the manufacturers instructions. This way, two non-overlapping probes against mGFRα3 were available for in situ analysis.

For tissue preparations, E16 mice were briefly washed in fresh ice cold phosphate-buffered saline (PBS) for a few minutes, transferred to ice-cold 4% paraformaldehyde (Lillys) and left over night at 4° C. for fixation. The next day, mice embryos were transferred to cold 30% sucrose in potassium phosphate buffered saline (KPBS) and left for 24 hours. Finally, embryos were embedded in Tissue-Tek, cut in 10 μm transverse cryosections and slides stored at −20° C. In situ hybridization was done according to Henrique et al. (1995) Nature, 787-790 with minor modifications. Briefly, frozen sections were thawed and postfixed in 4% paraformaldehyde in PBS for 10 minutes. Sections were pretreated for hybridisation as follows: 2×5 minutes in PBS; 5 minutes in 70% ethanol; 2 minutes in 96% ethanol. In parallel, DIG-labelled RNA probes were diluted in hybridisation buffer (50% formamide, 1×Denhardts solution, 10% dextran sulphate, 1 mg/ml tRNA and 1×SALTS according to Henrique et al. (1995) Nature, 787-790) and denatured for 10 minutes at 80° C. Slides were incubated overnight at 55° C. with 300 μL denatured probe in a humidified chamber. After hybridisation, slides were washed as follows: 1×15 minutes washing buffer (1×SSC; 50% formamide; 0.1% Tween-20) at 65° C.; 3×30 min at 65° C. in washing buffer; 2×30 minutes at room temperature in 1×MABT (100 mM maleic acid; 150 mM NaCl; 0.1% Tween-20).

To stain for DIG-labelled probe hybridized to GFRα3 mRNA, sections were blocked at room temperature for at least one hour in MABT with 2% blocking reagent (Roche) and 20% heat-inactivated goat serum (Zymed). Anti-DIG antibody (1:2000) was applied overnight in the same solution at room temperature. Slides were washed thoroughly in MABT for 5×10 minutes and rinsed with alkaline phosphatase staining buffer (100 mM NaCl; 100 mM Tris-HCl at pH 9.5; 50 mM MgCl2; 0.1% Tween-20) for 2×10 minutes. Staining was done at room temperature in alkaline phosphatase staining buffer containing 225 μg/mL NBT and 350 μg/mL BCIP. Before mounting with Pertex, the stained slides were rinsed twice in water and dried. Digital images were collected using an Olympus BX61 microscope and an Olympus DP50 digital camera.

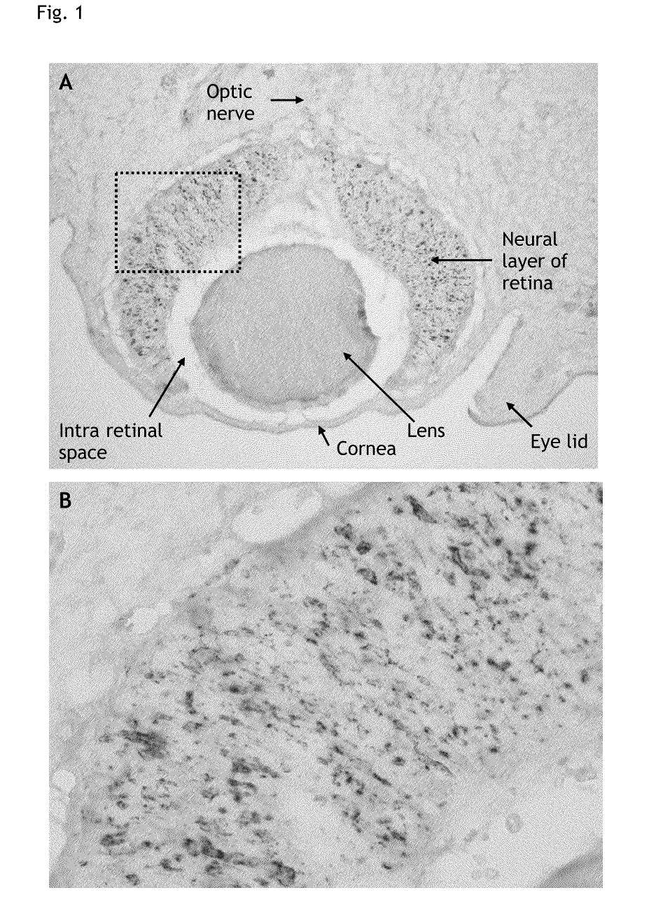

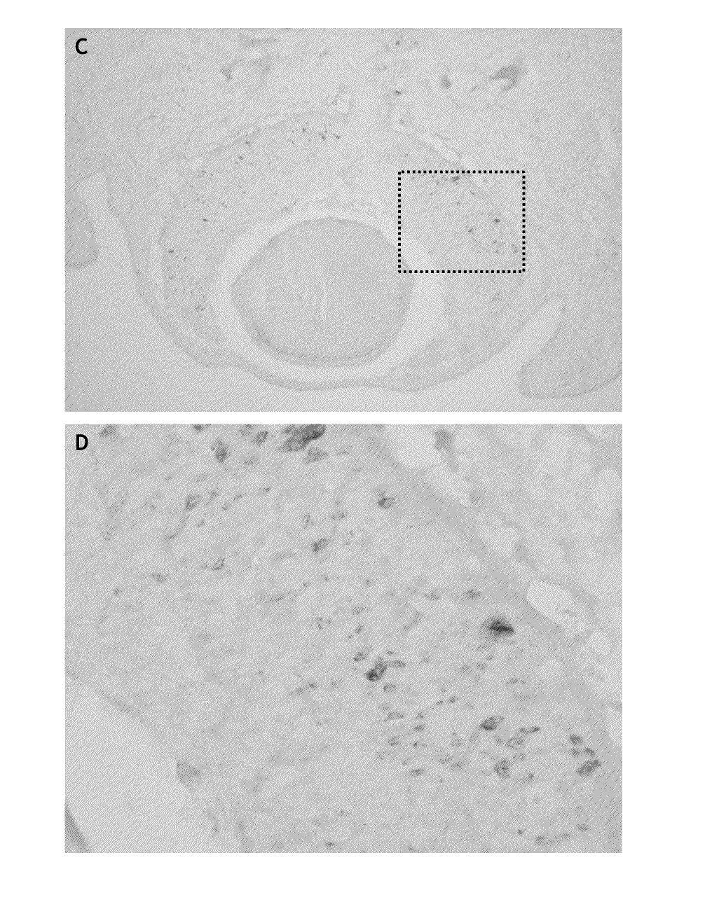

Results of the in situ hybridizations are shown in FIG. 1A-D. Using the GFRα3_{—}634-943 probe, a very intense staining for alkaline phosphatase is observed in the neural layer of the E16 murine retina (FIGS. 1A and B). Using the non-overlapping GFRα3_{—}113-377 probe on similar sections, results in a very similar expression pattern as seen from FIG. 1C-D. Even though the signal from the latter probe is weaker; many cells are seen with clear cytoplasmic staining. For both probes, note that there is no staining outside the retina. Also it should be noted that there is no signal from negative control slides without probe, ruling out endogenous alkaline phosphatase activity (data not shown). Therefore, having two independent probes showing virtually the same expression profiles are a good indication that GFRα3 is expressed in the mouse retina at E16. Both probes showed GFRα3 expression in the organs reported consistently in literature to have abundant expression (e.g. dorsal root ganglion) in mice embryos.

### Example 2

### Quantitative Expression Analysis

Quantitative real time PCR was used to investigate GFRα3 expression in human adult retina (BD, 636579). To be able to estimate biologically relevant expression levels, cDNA prepared from dorsal root ganglions (DRG) were used as a positive control (BD, 636150). High GRFα3 expression in DRG has been reported in several studies (Orozco et al., 2001, Eur. J. Neurosci., 13(11):2177; Naveilhan et al., 1998, Proc Natl Acad Sci, 95:11295; Baloh et al., 1998, Proc Natl Acad Sci, 95:5801). Corneal material was also included in the analysis to investigate whether GFRα3 expression in the eye is limited to the retina. Human corneal dissections were kindly donated by Dr. Ingrid Florén Ögonkliniken, Universitetssjukhuset, Lund, Sweden. After dissection, tissues were instantly frozen in liquid nitrogen and stored at −80° C. Subsequently, total RNA was extracted using Trizol (Invitrogen) according to manufacturers instructions. To concentrate RNA and to remove traces of chromosomal DNA, RNeasy on column DNase digestion with RNase-Free DNase was used (QIAgen) and RNA subsequently eluted in ultra pure H₂O. For all samples, aliquots of 2.5 μg RNA was used as template for cDNA synthesis with an RNAseH deficient reverse transcriptase derived from MoMLV (SuperScript) and a poly-dT primer.

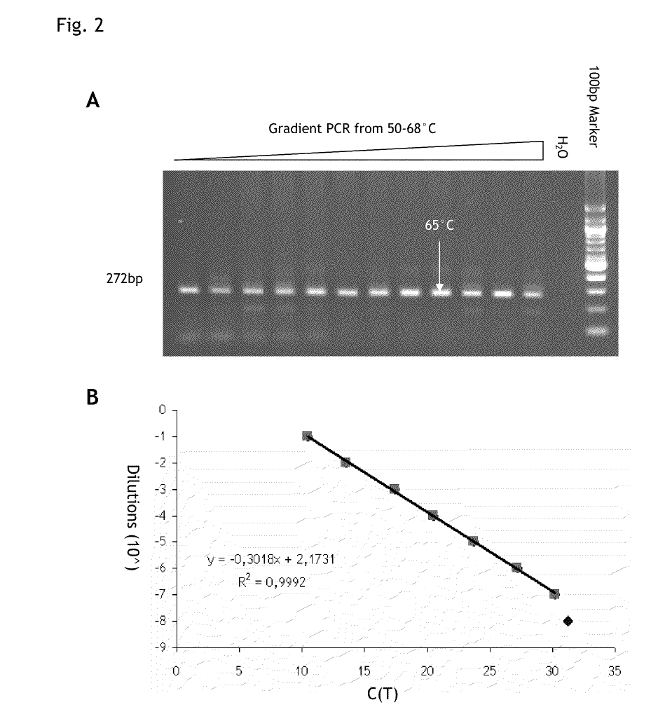

To detect GFRα3, a 5′ primer (5′-GGCAGCACAGCAACTCAGGAA-3′) annealing at bp 264 and a 3′ primer (5′-GCAGCCGGTCACACTTGTCAT-3′) annealing at bp 535 in GenBank sequence NM_{—}001496 was used amplifying a 272 bp fragment. The primer pair is intron spanning excluding a signal from genomic DNA. The optimal annealing temperature was determined by gradient RT-PCR (PTC-225, MJ Research) with cDNA prepared from Universal Human Reference RNA (Stratagene) as template. As can be seen from FIG. 2A one specific 272 bp band was produced at all temperatures and there was no signal from the negative H₂O control. The PCR product was subsequently isolated from the gel using the GENECLEAN II Kit (Qbiogene Inc) and serially diluted over eight decades to be used for generation of a standard curve (FIG. 2B). The standard curve was used to verify that crossing-point values (C(T)) were within the exponential range of the PCR reaction and to calculate final expression levels.

Using approximately 20 ng of each cDNA sample as template, Q-PCR was done in duplicates in an Opticon-2 thermocycler (MJ Research, Waltham, Mass.), using LightCycler-FastStart DNA Master SYBR Green I kit (Roche). All amplifications were performed in a total volume of 10 μl containing 3 mM MgCl2, 12% sucrose and 1× reaction buffer included in the LightCycler kit. The PCR cycling profile consisted of a 10 minutes pre-denaturation step at 98° C. followed by 35 three-step cycles; at 98° C. for 10 seconds, 65° C. for 20 seconds and finally at 72° C. for 20 seconds. Get electrophoresis and melting curve analysis was used to verify that a single PCR product of the predicted size was generated. To be able to normalize the samples and as a control for the integrity of the input RNA, all cDNA samples were subjected to real-time PCR using primers for glyceraldehyde-3-phosphate dehydrogenase (GAPDH, 5′-GGAAGGTGAAGGTCGGAGTCAA-3′ and 5′-GATCTCGCTCCTGGAAGATGGT-3′).

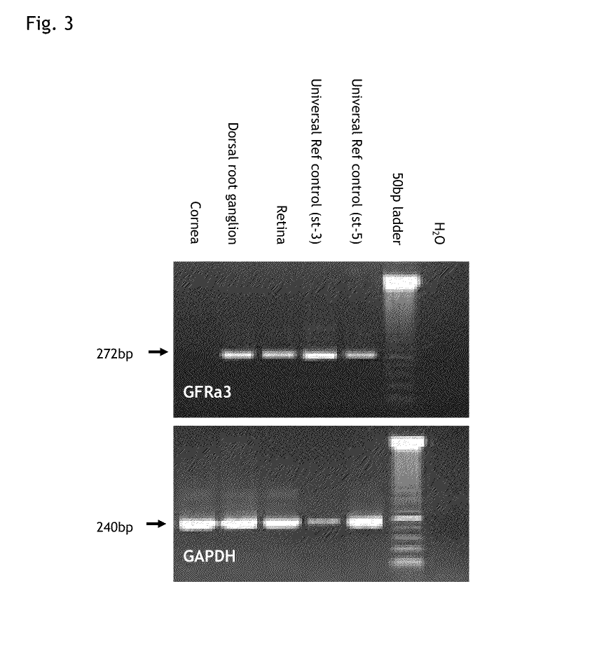

As seen in Table 1, C(T) values for GAPDH only differs 2-3 cycles, indicating that the cDNA is intact and equal amounts have been used for the analysis. This is also evident from FIG. 3 showing GAPDH bands of equal intensity for cornea, DRG and retina. In contrast, evident from both Table 1 and FIG. 3, GFRα3 is only expressed in retina and DRG but not in cornea. Importantly, the expression Level of GFRα3 in retina is comparable to the level in DRG and therefore at a biological relevant level.

TABLE 1
C(T) values for amplification of GAPDH and
GFRa3 fragment from Cornea, DRG and retina cDNA

| | C(T) for GAPDH | C(T) for GFRa3 |
|---|---|---|
| Cornea | 16.6/17.2 | 34.6/32.8 |
| Dorsal Root Ganglion | 16.3/16.6 | 21.4/21.4 |
| Retina | 14.5/14.5 | 22.5/22.2 |

There are published data on GDNF effects on photoreceptor degeneration and ret expression in the retina (Frasson et al., 1999 IOVS 40, 2724-2734). Furthermore, Neublastin has been shown to be a promising candidate for the treatment of pain mediated by its signalling in GFRα3/Ret expressing sensory neurons in the DRG. Thus our expression data on GFRα3 in the retina shown in this and the previous example together with the aforementioned published data indicate that Neublastin is a potential therapeutic candidate for the treatment of retinal disorders. Testing in retinal explant cultures of a photoreceptor degeneration model or in an excitotoxicity model may be used to verify this function.

### Example 3

### GFRα3 Immunolocalisation in Retina Histology

Pregnant Sprague-Dawley rats, MolTac:SD were sacrificed and E15 rat embryos were dissected out, washed in cold PBS and fixed in 4% paraformaldehyde in 0.1 M phosphate buffer (pH 7.4) for 24 h. Embryos were then transferred into 30% sucrose/0.1 M phosphate buffer for 48 h. Free-floating 50-μm sections were cut on a cryostat and used for GFRα3 immunohistochemistry. In brief, sections were incubated over night with goat-anti-mouse GFRα3 antibody (R&D Systems, AF2645) diluted 1:1000 in phosphate buffered saline with 2% Normal Horse Serum and 0.3% Triton X-100. Sections were subsequently incubated for two hours with anti-goat biotinylated secondary antibody (Jackson Immunoresearch, Trichem, DK) diluted 1:200 and followed by avidin-biotin complex (ABC) kit according to manufacturer's instructions (Vector Laboratories, USA). Finally, the color reaction was developed using 3′3′-diaminobenzidine as chromogen.

### Results

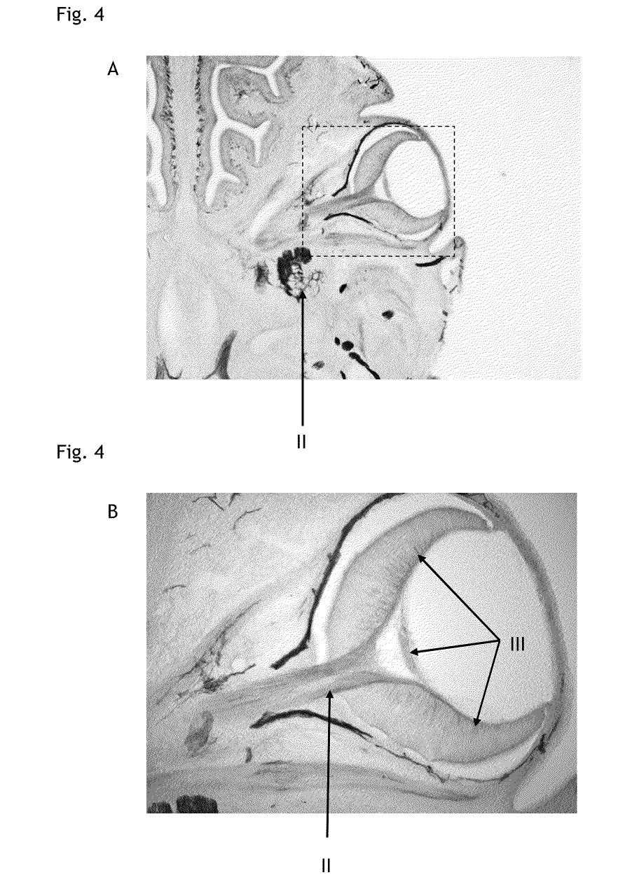

Representative results are shown in FIGS. 4A and 4B. Intense GFRα3 immunostaing was shown in trigemina (arrow I) and other nerve fiber structures in the E15 rat. In the retina, GFRα3 immunoreactive staining was observed in nerve fiber structures in the retinal ganglion cell layer (arrows II) and in the optic nerve (arrow III). The data confirm the presence of GFRα3 protein in the retina as exemplified by rat embryonal eyes.

1-19. (canceled)

20. A method of treatment of a retinal disorder said method comprising administering to an individual in need thereof a therapeutically effective amount of a GFRα3 agonist.

21. The method of claim 20, wherein said GFRα3 agonist activates RET through forming a complex together with GFRα3.

22. The method of claim 20, wherein said administration results in enhanced survival of retinal cells.

23. The method of claim 20, wherein said administration results in neurite outgrowth of retinal cells.

24. The method of claim 20, wherein said administration results in differentiation of retinal cells.

25. The method of claim 20, wherein the disorder is selected from the group consisting of inherited retinal dystrophies, age-related macular degeneration and other maculopathies, and retina detachment.

26. The method of claim 25, wherein the retinal dystrophy is retinitis pigmentosa.

27. The method of claim 20, wherein the retinal disorder involves retinal ischemic injury.

28. The method of claim 20, wherein the retinal disorder involves retinal degeneration.

29. The method of claim 20, wherein the retinal disorder is glaucoma.

30. The method of claim 20, wherein said individual is a human being.

31. The method of claim 20, wherein the GFRα3 agonist is Neublastin.

32. The method of claim 31, wherein Neublastin is administered as a Neublastin polypeptide, a polynucleotide encoding a Neublastin polypeptide, an expression vector encoding a Neublastin polypeptide, or a composition of cells secreting a Neublastin polypeptide.

33. The method of claim 32, wherein the Neublastin polypeptide comprises a polypeptide having an amino acid sequence having at least 70% sequence identity to SEQ ID NO 13.

34. The method of claim 32, wherein the Neublastin polypeptide is selected from the group consisting of human mature Neublastin or an N-terminally truncated human Neublastin.

35. The method of claim 32, wherein the Neublastin polypeptide has the amino acid sequence of SEQ ID NO 12 (NBN104).

36. The method of claim 32, wherein the Neublastin polypeptide is a dimer.

37. The method of claim 32, wherein the Neublastin polypeptide is glycosylated.

38. The method of claim 32, wherein the Neublastin polypeptide is polymer-conjugated, preferably wherein the polymer is PEG, more preferably wherein the polymer-conjugated Neublastin polypeptide is glycosylated.

39. The method of claim 32, wherein the Neublastin polypeptide is encoded by a gene therapy vector, preferably wherein said vector is an AAV vector.

40. The method of claim 32, wherein the Neublastin polypeptide is secreted from a composition of cells, encapsulated in a device having a semi-permeable membrane allowing the passage of Neublastin and protecting the encapsulated cells from the immune system of the individual to be treated.

41. The method of claim 40, wherein the encapsulated cells comprise ARPE-19 cells

42. The method of claim 39, wherein the expression construct encoding the Neublastin polypeptide does not encode a functional Neublastin prodomain.

43. The method of claim 20, wherein the individual is a human being.

44. A method of enhancing the survival of a retinal cell comprising contacting said cell with a GFRα3 agonist.

45. A method of differentiating a retinal cell comprising contacting said cell with a GFRα3 agonist.

46. The method of claim 45, wherein the differentiation is neurite outgrowth.

47. A method of providing in vitro trophic support to retinal cells, comprising contacting retinal cells with a GFRα3 agonist in vitro.

48. The method of claim 40, wherein the expression construct encoding the Neublastin polypeptide does not encode a functional Neublastin prodomain.
